(19) United States
(12) Patent Application Publication
(10) Pub. No.: US 2016/0362688 A1
(43) Pub. Date: Dec. 15, 2016

(54) COMPOSITIONS AND METHODS OF USING MICRORNA INHIBITORS

(71) Applicant: Thomas Jefferson University, Philadelphia, PA (US)

(72) Inventors: Eric Wickstrom, Philadelphia, PA (US); Yuan-Yuan Jin, Haddonfield, NJ (US)

(21) Appl. No.: 15/114,128

(22) PCT Filed: Feb. 12, 2015
(86) PCT No.: PCT/US15/15681

§ 371 Date: Jul. 26, 2016

Related U.S. Application Data
(60) Provisional application No. 61/938,776, filed on Feb. 12, 2014.

(51) Int. Cl. C12N 15/113

(52) U.S. Cl. CPC C12N 15/113; C12N 2310/113; C12N 2310/3181; C12N 2310/3231; C12N 2310/341

(57) ABSTRACT

The present invention provides compositions and methods of making and using microRNA inhibitors. In a particular embodiment, the invention features compositions and methods useful for the treatment of diseases, including neoplasia (e.g., breast cancer).

### CROSS REFERENCE TO RELATED APPLICATIONS

This application is a 35 U.S.C. §371 national phase application from, and claims priority to, International Application No. PCT/US2015/015681 filed Feb. 12, 2015, and published under PCT Article 21(2) in English, which claims priority under 35 U.S.C. §119(e) to U.S. Provisional Application No. 61/938,776, filed Feb. 12, 2014, all of which application is incorporated herein by reference in its entirety.

### STATEMENT OF RIGHTS TO INVENTIONS MADE UNDER FEDERALLY SPONSORED RESEARCH

This invention was made with government support under Grant No. CA148565 awarded by the National Institutes of Health. The government has certain rights in the invention.

### BACKGROUND OF THE INVENTION

MicroRNAs (miRNAs; miRs) play crucial roles in regulating cell division, differentiation and proliferation. Studies have suggested that miRNAs are encoded in more than 1% of all human genes (Lim et al., 2003, Science 299(5612):1540), and they target more than 30% of human genes (Lewis et al., 2005, Cell 120(1):15-20). miRNAs have been implicated in a number of biological processes, including reductions in levels of tumor suppressor proteins such as PTEN and PDCD4 in triple negative breast cancer (TNBC) cells.

miRNAs are non-coding RNAs that inhibit translation of mRNAs by disrupting target mRNA translation or through targeting mRNA degradation (Bartel, 2004, Cell 116(2):281-97). Biogenesis of miRNAs initiates in the nucleus where primary miRNAs are transcribed by RNA polymerase II. Primary miRNAs are then processed by Drosha and its cofactor DGCR8 to produce shorter hairpin precursor miRNAs (Lee et al., 2003, Nature 425(6956):415-9). Pre-miRNAs are exported to the cytoplasm by exportin 5 and cleaved by Dicer, removing the loop, to yield mature double-stranded miRNAs. One strand of the double-stranded miRNA contains unstable hydrogen bonding at its 5′ end, and is preferentially incorporated into the RNA-induced silencing complex (RISC) as a guide strand (Khvorova et al., 2003, Cell 115(2):209-16). The other strand, called a passenger strand, is assumed to dissociate from RISC and be degraded. However, some activity of passenger strands has been asserted (Mah et al., 2010, Crit Rev Eukaryot Gene Expr 20(2):141-8). The results described herein indicate that the miR-17-3p passenger strand targets PTEN and PDCD4 mRNAs, contradicting the conventional wisdom.

Several regulatory mechanisms of miRNAs have been proposed. Translational repression by miRNAs can be achieved by guide strand miRNAs binding to targets in the 3′UTR of mRNAs through imperfect complementarity mediated by RISC (Bartel, 2004, Cell 116(2):281-97). RISC also binds to the open reading frame of target mRNAs with perfect or near perfect complementarity, and results in mRNA cleavage by the endonuclease active site of Ago2. Ago2 can also bind to the m⁷G cap of mRNA to shield the binding site for the translation initiation factor eIF4E (Khoshnaw et al., 2009, J Clin Pathol 62(5):422-8). miRNA-bound mRNAs and associated RISC proteins are sometimes stored in P-bodies to prevent translational repression, unless released under stress to re-enter polysomes (Lynam-Lennon et al., 2009, Biol Rev Camb Philos Soc 84(1):55-71).

A given miRNA may have multiple different mRNA targets, and a given target might similarly be targeted by multiple miRNAs. miRNAs play crucial roles in regulating cell division, differentiation and proliferation in normal or diseased cells. Studies have suggested that miRNAs are encoded in more than 1% of all human genes (Lim et al., 2003, Science 299(5612):1540), and they target more than 30% of human genes (Lewis et al., 2005, Cell 120(1):15-20). Conserved seed pairing, often flanked by adenosines, indicates that thousands of human genes are microRNA targets. In terms of cancer, there are oncogenic miRNAs and tumor suppressor miRNAs (Cimmino et al., 2005, Proc Natl Acad Sci USA 102(39):13944-9. PMID: 16166262; O'Donnell et al., 2005, Nature 435(7043):839-43. PMID: 15944709; Johnson et al., 2005, Cell 120(5):635-47. PMID: 15766527). Oncogenic miRNAs target tumor suppressor genes, while tumor suppressor miRNAs target oncogenes. Overexpression of oncogenic miRNAs decreases the protein level of tumor suppressors, allowing tumorigenesis. Depletion of tumor suppressor miRNAs results in overexpression of oncogenes, leading to continuous cell proliferation and division (Caldas et al., 2005, Nat Med 11(7):712-4).

Triple negative breast cancer (TNBC) lacks the estrogen receptor (ER), progesterone receptor (PR), or human epidermal growth factor 2 (Her2) molecular targets, resulting in a poor prognosis with few treatment options. TNBC usually responds at first to chemotherapy, but has a short time to relapse, with few treatment options (Bosch et al., 2010, Cancer Treat Rev 36(3):206-15). The lack of ER/PR precludes use of antiestrogens. The lack of Her2 negates treatment with Her2 inhibitors. Tumor suppressor proteins such as PTEN (Depowski et al., 2001, Mod Pathol 14(7):672-676) and PDCD4 (Frankel et al., 2008, J Biol Chem 283(2):1026-33) are reduced in TNBC cells. Loss of suppressor activity allows increased TNBC cell proliferation, survival, microfilament destabilization, metastatic transformation, and invasion of surrounding tissues and blood vessels.

The dysregulation of homeostasis in many cancers has been related to changes in the expression of miRNAs (Iorio et al., 2005, Cancer Res 65(16):7065-7070). miRNA expression levels more accurately represent the functional activity of the gene compared with the expression levels of mRNAs (Rosenfeld et al., 2008, Nat Biotechnol 26(4):462-9). miRNA expression profiling helps to differentiate cancer from normal tissues and can effectively categorize even poorly differentiated cancer tissues (Lu et al., 2005, Nature 435(7043):834-8). miRNA expression could be used to classify human breast tumors, predict prognosis and distinguish cancer tissue from adjacent normal tissue (Iorio et al., 2005, Cancer Res 65(16):7065-7070). Numerous regulatory miRNAs of breast cancer cell proliferation and metastasis are being studied (Pencheva et al., 2013, Nat Cell Biol 15(6):546-554).

Oncogenic miRNAs (oncomiRs) are microRNAs associated with cancer by suppressing the translation of tumor suppressor genes. The dysregulation of such microRNAs (oncomiRs) has been associated with specific cancer forming (oncogenic) events, including carcinogenesis, malignant transformation, and metastasis. Many different oncomiRs have been identified in numerous types of human cancers. OncomiRs may be at increased or decreased levels within cancerous tissue. Some oncomiRs derive from oncogenes, in that overexpression of the gene leads to increased survival and/or decreased cell death of neoplastic cells. Such oncomiRs may cause cancer by down-regulating genes (e.g., tumor suppressors and/or proteins that regulate the cell's life cycle) by both translational repression and mRNA destabilization mechanisms. Other oncomiRs regulate tumor suppressor activity in a normal cell, so that underexpression of this type of oncomir leads to neoplastic cell growth and/or proliferation.

OncomiRs target tumor suppressor genes, while tumor suppressor miRNAs target oncogenes. Overexpression of oncomiRs decreases the protein level of tumor suppressors, allowing tumorigenesis. Depletion of tumor suppressor miRNAs results in overexpression of oncogenes, leading to continuous cell proliferation and division (Caldas et al., 2005, Nat Med 11(7):712-4). The oncomiRs miR-17-5p (Yu et al., 2008, J. Cell Biol. 182(3):509-517) and miR-21-5p (Lu et al., 2008, Oncogene 27(31):4373-9. PMID: 1837292) are typically overexpressed in TNBC cells, which particularly show activation of the entire miR-17-92 cluster (Farazi et al., 2011, Cancer Research 71(13):4443-4453). miR-17-5p (Shan et al., 2013, J Cell Sci 126(Pt 6):1517-30). miR-21-5p (Meng et al., 2007, Gastroenterology 133(2):647-58) inhibits the translation of PTEN mRNA. miR-21-5p inhibits the translation of PDCD4 mRNA (Frankel et al., 2008, J Biol Chem 283(2):1026-33). miR-17-5p and miR-21-5p are also found in circulating exosomes (Valadi et al., 2007, Nat Cell Biol 9(6):654-9), with unknown consequences.

Many kinds of cancer overexpress miR-17-5p, a member of the miR-17-92 cluster, a family of homologous miRNAs with genomic positions on chromosomes X, 13 and 7 (He et al., 2005, Nature 435(7043):828-33). The cluster located on chromosome 13 is often amplified in B-cell lymphoma (Ota et al., 2004, Cancer Res 64(9):3087-95), and miRNAs from the chromosome 13 cluster are generally up-regulated in various cancers, including breast, lung, colon, pancreas, prostate, and gastric cancer (Volinia et al., 2006, Proc Natl Acad Sci USA 103(7):2257-61; Petrocca et al., 2008, Cancer Cell 13(3):272-86). This gene cluster is transcribed as a single primary-miRNA and then processed to produce six single mature miRNA molecules: miR-17-5p, miR-18, miR-19a, miR-20, miR-19b-1 and miR-92-1 (Tanzer et al., 2004, J Mol Biol 339(2):327-35), with five of them overexpressed in cell lines having this amplified gene cluster (He et al., 2005, Nature 435(7043):828-33). Caloric restriction (CR) and ionizing radiation (IR) down-regulate members of the miR-17˜92 cluster in mouse 4T1 tumor models of triple negative breast cancer, decreasing their metastatic activities by suppressing extracellular matrix (ECM) mRNAs that exhibit miR-17-5p binding sites (Jin et al., 2014, Breast Cancer Res Treat 146(1):41-50). Among the seven members of the miR-17˜92 cluster, the guide strand miR-17-5p is predominantly responsible for promoting migration and invasion of metastatic cells, targeting the mRNAs of tumor suppressor genes, such as PDCD4 and PTEN (Xiao et al., 2008, Nat Immunol 9(4):405-14). miR-17-5p is significantly up-regulated in mesenchymal MDA-MB-231 cells compared to the noninvasive luminal MCF7 cells, and contributes to invasiveness and migratory behavior (Li et al., 2011, Breast Cancer Res Treat 126(3):565-75). As a passenger strand, miR-17-3p has been reported to target vimentin mRNA in hepatocellular carcinoma (Shan et al., 2013, J Cell Sci 126(Pt 6):1517-30).

miR-21-5p guide strand expression is upregulated in pancreatic cancer, correlating with increased proliferation and metastasis (Roldo et al., 2006, J Clin Oncol 24(29):4677-84). The ability of miR-21-5p to discriminate between chronic pancreatitis and pancreatic cancer further confirmed its role in carcinogenesis (Bloomston et al., 2007) Jama 297(17):1901-8). miR-21-5p is overexpressed in TNBC, suggesting a role in tumorigenesis (Frankel et al., 2008, J Biol Chem 283(2):1026-33; Lu et al., 2008, Oncogene 27(31):4373-9; Iorio et al., 2005, Cancer Res 65(16):7065-7070). miR-21-3p has been reported to target NAV3 mRNA in cisplatin-resistant ovarian cancer cells (Pink et al., 2015, Gynecol Oncol.). The oncogenic characteristic of miR-21-5p, the guide strand, is reflected through its upregulated expression in pancreatic cancer, correlating with increased proliferation and metastasis (Roldo et al., 2006, J Clin Oncol 24(29):4677-84). The ability of miR-21-5p to discriminate between chronic pancreatitis and pancreatic cancer further confirmed its role in carcinogenesis (Bloomston et al., 2007, JAMA 297(17):1901-8). miR-21-5p is overexpressed in breast cancer, suggesting a role in tumorigenesis (Frankel et al., 2008, J Biol Chem 283(2):1026-33; Lu et al., 2008, Oncogene 27(31):4373-9; Iorio et al., 2005, Cancer Res 65(16):7065-7070). Outside of cancer, miR-21-5p encourages cell proliferation to replace lost or dead cells, as in liver regeneration after ethanol insult (Dippold, R. P., et al., 2012, Am J Physiol Gastrointest Liver Physiol 303(6):G733-743).

PTEN protein is a tumor suppressor protein, negatively regulating cell proliferation and survival. Impairment of PTEN regulation is thought to play a role in oncogenic transformation (Maehama, 2007, Biol Pharm Bull 30(9):1624-7). miR-17-5p was found overexpressed and targeted to the PTEN 3′-UTR in glioblastoma cells deprived of nutrition or treated with chemotherapeutics (Li et al., 2012, Oncotarget 3(12):1653-68). The significance of miR-17-5p in breast cancer remains controversial. miR-21-5p has been identified as a potential regulator of the PTEN gene in hepatocellular carcinoma (HCC) (Meng et al., 2007, Gastroenterology 133(2):647-58). The regulatory region on PTEN mRNA was demonstrated to reside at the 3′-UTR using a luciferase reporter construct containing a fragment of the 3′-UTR of PTEN mRNA corresponding to the putative miR-21-5p binding sequence (Meng et al., 2007, Gastroenterology 133(2):647-58). In breast cancer, introduction and inhibition of miR-21-5p caused only subtle changes in PTEN protein expression, suggesting that functional targets of miR-21 may differ in different cell/tissue types (Lynam-Lennon et al., 2009, Biol Rev Camb Philos Soc 84(1):55-71).

Tumor suppressor protein PDCD4 is overexpressed during apoptosis (Frankel et al., 2008, J Biol Chem 283(2):1026-33). Its downregulation in lung and colorectal cancer was associated with poor survival prognosis (Chen et al., 2003, J Pathol 200(5):640-6; Mudduluru et al., 2007, Cancer 110(8):1697-707). The seed region on PDCD4 mRNA for miR-21-5p binding resides within the 3′-UTR (Asangani, I. A., Rasheed, S. A., Nikolova, D. A., Leupold, J. H., Colburn, N. H., Post, S., and Allgayer, H. (2008) MicroRNA-21 (miR-21) post-transcriptionally downregulates tumor suppressor PDCD4 and stimulates invasion, intravasation and metastasis in colorectal cancer. Oncogene 27(15):2128-36). In MCF-7 breast cancer cells, PDCD4 protein is specifically regulated by miR-21-5p interacting with the seed region of the PDCD4 mRNA 3′-UTR (Frankel et al., 2008, J Biol Chem 283(2):1026-33). Overexpression of miR-21-5p increased MCF-7 breast cancer cell invasion, indicating a role in metastatic transformation (Lu et al., 2008, Oncogene 27(31):4373-9).

A variety of miRNA profiling studies have reported differentially expressed miRNA passenger strands, such as miR-9-3p, miR-18-3p, miR-29c-3p, miR-126-3p, miR-146-3p, miR-199-3p, miR-223-3p, and miR-363-3p in a spectrum of disease states (Mah et al., 2010, Crit Rev Eukaryot Gene Expr 20(2):141-8). Additional possibilities are noted in miRBase (www.mirbase.org).

Accordingly, miRNAs represent a relatively new class of therapeutic targets for the treatment of cancer and other other diseases involving miRNA regulation. miRNA function may be targeted therapeutically by antisense oligonucleotides or by oligonucleotides that mimic miRNA function. However, targeting miRNAs therapeutically with oligonucleotide-based agents poses several challenges, including RNA-binding affinity and specificity. Antisense oligonucleotides that target miRNAs while minimizing off-target effects have the potential to provide therapeutic outcomes.

At present, no effective treatment exists for triple negative breast cancer (TNBC). New methods of treatment for TNBC, other neoplasms and diseases involving miR regulation are urgently required. The present invention addresses these unmet needs in an unexpected fashion.

### SUMMARY OF THE INVENTION

The present invention features compositions and methods for specifically binding and/or inhibiting the activity of microRNAs, while decreasing off-target effects. Such inhibitors of miRNAs may be used for the treatment of diseases, including neoplasms (e.g., triple negative breast cancer).

In one aspect, the invention provides an isolated inhibitory nucleic acid that is fully complementary to at least 50% of a microRNA (miRNA) strand, but no more than 75% (e.g., no more than 70%, 65%, 60%, 55%) of the miRNA strand, starting at the 5′ region of the miRNA strand.

In another aspect, the invention provides a method for treating neoplasia in a subject involving administering to the subject an effective amount of the an inhibitory nucleic acid of any aspect of the invention that binds to miR-17-5p or miR-21-5p.

In another aspect, the invention provides a method of decreasing binding of an miRNA to an mRNA in a cell involving administering to the cell an inhibitory nucleic acid that is fully complementary to at least 50% of a microRNA (miRNA) strand, but no more than 75% (e.g., no more than 70%, 65%, 60%, 55%) of the miRNA strand, starting at the 5′ region of the miRNA strand.

In various embodiments of any aspect delineated herein, the miRNA strand is a guide strand or passenger strand. In various embodiments, the inhibitory nucleic acid binds the guide strand or passenger strand (e.g., targets the guide strand or passenger strand). In some embodiments, the inhibitory nucleic acid is fully identical to at least 50% of an miRNA strand, but no more than 75% (e.g., no more than 70%, 65%, 60%, 55%) of the miRNA strand, starting at the 3′ region of the miRNA strand. In some embodiments, the inhibitory nucleic acid does not bind or minimizes binding to an mRNA targeted by the miRNA. In particular embodiments, the inhibitory nucleic acid sequence is selected to eliminate or minimize binding to an mRNA targeted by the miRNA. In certain embodiments, the inhibitory nucleic acid specifically binds the seed region of the targeted miRNA strand (e.g., the guide strand or passenger strand). In particular embodiments, the inhibitory nucleic acid includes up to three bases of the seed region of the opposite miRNA strand, that is not targeted. In some embodiments, the inhibitory nucleic acid excludes the sequence of the seed region of the opposite miRNA strand, that is not targeted.

In various embodiments of any aspect delineated herein, the inhibitory nucleic acid is DNA or RNA. In various embodiments, the inhibitory nucleic acid comprises one or more modifications selected from phosphorothioate, morpholino phosphoramidate, methylphosphonate, boranophosphate, locked nucleic acid, peptide nucleic acid, 2′-fluoro, 2′-amino, 2′-thio, or 2′-O-alkyl.

In various embodiments of any aspect delineated herein, the miRNA is miR-17 or miR-21. In various embodiments, the mRNA is a PTEN or PDCD4 mRNA. In certain embodiments, the inhibitory nucleic acid binds to miR-17-5p or miR-21-5p. In particular embodiments, the inhibitory nucleic acid contains the nucleic acid sequence 5′-GTAAGCACTTTG-3′(SEQ ID NO: 1) and binds miR-17-5p. In particular embodiments, the inhibitory nucleic acid contains the nucleic acid sequence 5′-TCTGATAAGCTA-3′(SEQ ID NO: 2) and binds miR-21-5p. In some embodiments, the inhibitory nucleic acid does not bind or minimizes binding to a PTEN or PDCD4 mRNA.

In various embodiments of any aspect delineated herein, the neoplasm is breast cancer, including triple negative breast cancer. In various embodiments of any aspect delineated herein, the cell is a breast cancer or triple negative breast cancer cell.

### BRIEF DESCRIPTION OF THE DRAWINGS

The following detailed description of preferred embodiments of the invention will be better understood when read in conjunction with the appended drawings. For the purpose of illustrating the invention, they are shown in the drawings embodiments which are presently preferred. It should be understood, however, that the invention is not limited to the precise arrangements and instrumentalities of the embodiments shown in the drawings.

FIG. 1 are graphs depicting qPCR results that show >95% knockdown of miR-17-5p (left) and miR-21-5p (right) in MDA-MB-231 cells treated with 50 nM LNAs. LNAs were transfected via lipofectamine 2000 in Opti-MEM.

FIG. 2 depicts miR-17-5p, miR-17-3p, and miR-21-5p targets found in PDCD4 3′-UTR.

FIG. 3 depicts identification of miR-17-5p, miR-17-3p, and miR-21-5p targets in PDCD4 and PTEN using a prediction program (rna22).

FIG. 4, comprising FIGS. 4A-4C, depicts Western Blot analysis of PTEN and PDCD4 protein levels. In FIG. 4A, a representative Western Blot is shown of PTEN (left) and PDCD4 (right) protein levels. In FIG. 4B, PDCD4 protein levels from several experiments were quantified. In FIG. 4C, PTEN protein levels from several experiments were quantified. Protein levels were determined by Western Blot analysis following 50 nM LNA knockdown of miR-17-5p in MDA-MB-231 cells for 6 hr. LNAs were transfected via lipofectamine 2000 in Opti-MEM.

FIG. 5, comprising FIGS. 5A-5C, depicts Western Blot analysis of PTEN and PDCD4 protein levels. In FIG. 5A, a representative Western Blot is shown of PTEN (left) and PDCD4 (right) protein levels. In FIG. 5B, PDCD4 protein levels from several experiments were quantified. In FIG. 5C, PTEN protein levels from several experiments were quantified. Protein levels were determined by Western Blot analysis following 1 PNA-peptide knockdown of miR-17-5p in MDA-MB-231 cells for 6 hr. PNA-peptides were endocytosed via IGF1R in Opti-MEM.

FIG. 6 depicts a molecular dynamic prediction of miR-17-3p passenger strand binding stably to nucleotides 3768-3789 in the 3′UTR of PTEN mRNA, in A-form helix. Extra bases and mismatched bases stay stacked within the helix, accommodated by backbone distortions.

FIG. 7 depicts the results of an miRBase search of miR-17-5p and miR-17-3p. Homologous sequences between miR-17-5p and miR-17-3p are highlighted in grey.

FIG. 8 depicts an Mfold calculation of miR-21-3p passenger strand binding to nucleotides 228-249 in the 3′UTR of PDCD4 mRNA. Calculated ΔG°=−10.5 kcal/mol in 0.1 M NaCl.

FIG. 9 is a graph depicting that caloric restriction reduced the expression of miR-17 and miR-20a in 4T1 tumor model measured under 4 different conditions: ad libitum feeding (AL), radiation (IR), caloric restriction (CR), and CR+IR.

FIG. 10 is a graph showing qPCR of miR-17-5p 12 hr hr post-transfection of MDA-MB-231 cells with anti-miR-17-5p. Results represent absolute values of miRNA/internal control U6 normalized to mock transfected. Values are the average of n=3 measurements±s.d.

FIG. 11 depicts Western Blot analysis of PTEN and PDCD4 protein levels at 48 hr post transfection with anti-miR-1′7-5p.

FIG. 12 depicts Western Blot analysis of PTEN and PDCD4 protein levels at 48 hr post transfection with anti-miR-17-3p.

FIG. 13 depicts Western Blot analysis of PTEN and PDCD4 protein levels at 48 hr post transfection with anti-miR-21-5p.

FIG. 14 depicts the structure of a PNA of the invention comprising Near infrared CN-1016-AEEA-PNA-AEEA-cyclo-D(Cys-Ser-Lys-Cys).

FIG. 15 depicts Western Blot analysis of PTEN and PDCD4 protein levels blocked by anti-miR-17 and anti-miR-21 PNAs. 1 μM anti-miR-17 PNA-d(CSKC), anti-miR-21 PNA-d(CSKC), or scrambled PNA-d(CSKC) was incubated 48 hr with MDA-MB-231 cells. Lysate proteins were analyzed by Western blot.

FIG. 16 depicts exemplary miRNA inhibitors designed in accordance with the invention. The stem-loop structure for each miRNA shows the complementarity between the guide strand (top sequence highlighted in grey) and the passenger strand (bottom sequence highlighted in grey). Each miRNA, which the inhibitor is designed for, is shown as the top sequence, whereas the sequence marked with dashed lines represents nucleotides that are complementary to the seed region of the other strand. The inhibitor sequence is shown as the bottom sequence, whereas the underlined part of the sequence represents possible extension of the inhibitor sequence to include several nucleotides into the seed sequence of the other strand. The shared targets between the guide and the passenger strands are predicted by miRWalk, DIANA-mT, miRanda, miRDB, PICTAR, PITA, rna22, and TargetScan with minimum of 6 seed pairing. Cancer association for selected miRNAs is summarized from miRCancer database.

FIG. 17 is a table listing miRNAs and miRNA inhibitors relevant for the present invention (SEQ ID NOs: 3-39).

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

By “microRNA” or “miRNA” or “miR” is meant a small non-coding RNA, which functions in transcriptional and/or post-transcriptional regulation of gene expression. In various embodiments, miRNAs have a hairpin structure comprising a duplex that is processed into a guide strand and a passenger strand.

“Pre-miRNA” or “pre-miR” means a non-coding RNA having a hairpin structure, which is the product of cleavage of a pri-miR by double-stranded RNA-specific ribonuclease.

“Pri-miRNA” or “pri-miR” means a non-coding RNA having a hairpin structure that is a substrate for double-stranded RNA-specific ribonuclease.

By the phrase “miRNA precursor” means a transcript that originates from a genomic DNA and that comprises a non-coding, structured RNA comprising one or more miRNA sequences. For example, in certain embodiments, a miRNA precursor is a pre-miRNA. In certain embodiments, a miRNA precursor is a pri-miRNA.

By “miR-17” is meant human miR-17, and is substantially identical to the nucleic acid sequence of GenBank Accession No. NR_029487, or a fragment thereof (SEQ ID NO: 40; and SEQ ID NO: 3 in FIG. 17). In one embodiment, an miR-17 has at least about 85% nucleic acid sequence identity to the sequence provided below:

| (SEQ ID NO: 40) | |
|---|---|
| 1 | gtcagaataa tgtcaaagtg cttacagtgc aggtagtgat atgtgcatct actgcagtga |
| 61 | aggcacttgt agcattatgg tgac |

By “miR-21” is meant human miR-21, and is substantially identical to the nucleic acid sequence of GenBank Accession No. NR_029493, or a fragment thereof (SEQ ID NO: 41; and SEQ ID NO: 19 in FIG. 17). In one embodiment, an miR-424 has at least about 85% nucleic acid sequence identity to the sequence provided below:

| (SEQ ID NO: 41) | |
|---|---|
| 1 | tgtcgggtag cttatcagac tgatgttgac tgttgaatct catggcaaca ccagtcgatg |
| 61 | ggctgtctga ca |

By “Phosphatase and tensin homolog (PTEN) polypeptide” is meant a polypeptide or fragment thereof having at least 85% amino acid identity to NCBI Accession No. NP_000305 and having phosphatase and/or tumor suppressor activity. An exemplary PTEN polypeptide sequence is provided below (SEQ ID NO: 42):

| | |
|---|---|
| 1 | mtaiikeivs rnkrryqedg fdldltyiyp niiamgfpae rlegvyrnni ddvvrfldsk |
| 61 | hknhykiynl caerhydtak fncrvaqypf edhnppqlel ikpfcedldq wlseddnhva |
| 121 | aihckagkgr tgvmicayll hrgkflkaqe aldfygevrt rdkkgvtips qrryvyyysy |
| 181 | llknhldyrp vallfhkmmf etipmfsggt cnpqfvvcql kvkiyssnsg ptrredkfmy |
| 241 | fefpqplpvc gdikveffhk qnkmlkkdkm fhfwvntffi pgpeetsekv engslcdqei |
| 301 | dsicsierad ndkeylvltl tkndldkank dkanryfspn fkvklyftkt veepsnpeas |
| 361 | sstsvtpdvs dnepdhyrys dttdsdpene pfdedqhtqi tkv |

By “PTEN nucleic acid molecule” is meant a polynucleotide encoding a PTEN polypeptide. An exemplary PTEN nucleic acid sequence is provided at NCBI Accession No. NM_000314. An exemplary PTEN mRNA transcript is provided below (SEQ ID NO: 43):

| | |
|---|---|
| 1 | cctcccctcg cccggcgcgg tcccgtccgc ctctcgctcg cctcccgcct cccctcggtc |
| 61 | ttccgaggcg cccgggctcc cggcgcggcg gcggaggggg cgggcaggcc ggcgggcggt |
| 121 | gatgtggcgg gactctttat gcgctgcggc aggatacgcg ctcggcgctg ggacgcgact |
| 181 | gcgctcagtt ctctcctctc ggaagctgca gccatgatgg aagtttgaga gttgagccgc |
| 241 | tgtgaggcga ggccgggctc aggcgaggga gatgagagac ggcggcggcc gcggcccgga |
| 301 | gcccctctca gcgcctgtga gcagccgcgg gggcagcgcc ctcggggagc cggccggcct |
| 361 | gcggcggcgg cagcggcggc gtttctcgcc tcctcttcgt cttttctaac cgtgcagcct |
| 421 | cttcctcggc ttctcctgaa agggaaggtg gaagccgtgg gctcgggcgg gagccggctg |
| 481 | aggcgcggcg gcggcggcgg cacctcccgc tcctggagcg ggggggagaa gcggcggcgg |
| 541 | cggcggccgc ggcggctgca gctccaggga gggggtctga gtcgcctgtc accatttcca |
| 601 | gggctgggaa cgccggagag ttggtctctc cccttctact gcctccaaca cggcggcggc |
| 661 | ggcggcggca catccaggga cccgggccgg ttttaaacct cccgtccgcc gccgccgcac |
| 721 | cccccgtggc ccgggctccg gaggccgccg gcggaggcag ccgttcggag gattattcgt |
| 781 | cttctcccca ttccgctgcc gccgctgcca ggcctctggc tgctgaggag aagcaggccc |
| 841 | agtcgctgca accatccagc agccgccgca gcagccatta cccggctgcg gtccagagcc |
| 901 | aagcggcggc agagcgaggg gcatcagcta ccgccaagtc cagagccatt tccatcctgc |
| 961 | agaagaagcc ccgccaccag cagcttctgc catctctctc ctcctttttc ttcagccaca |
| 1021 | ggctcccaga catgacagcc atcatcaaag agatcgttag cagaaacaaa aggagatatc |
| 1081 | aagaggatgg attcgactta gacttgacct atatttatcc aaacattatt gctatgggat |
| 1141 | ttcctgcaga aagacttgaa ggcgtataca ggaacaatat tgatgatgta gtaaggtttt |
| 1201 | tggattcaaa gcataaaaac cattacaaga tatacaatct ttgtgctgaa agacattatg |
| 1261 | acaccgccaa atttaattgc agagttgcac aatatccttt tgaagaccat aacccaccac |
| 1321 | agctagaact tatcaaaccc ttttgtgaag atcttgacca atggctaagt gaagatgaca |
| 1381 | atcatgttgc agcaattcac tgtaaagctg gaaagggacg aactggtgta atgatatgtg |
| 1441 | catatttatt acatcggggc aaatttttaa aggcacaaga ggccctagat ttctatgggg |
| 1501 | aagtaaggac cagagacaaa aagggagtaa ctattcccag tcagaggcgc tatgtgtatt |
| 1561 | attatagcta cctgttaaag aatcatctgg attatagacc agtggcactg ttgtttcaca |
| 1621 | agatgatgtt tgaaactatt ccaatgttca gtggcggaac ttgcaatcct cagtttgtgg |
| 1681 | tctgccagct aaaggtgaag atatattcct ccaattcagg acccacacga cgggaagaca |
| 1741 | agttcatgta ctttgagttc cctcagccgt tacctgtgtg tggtgatatc aaagtagagt |
| 1801 | tcttccacaa acagaacaag atgctaaaaa aggacaaaat gtttcacttt tgggtaaata |
| 1861 | cattcttcat accaggacca gaggaaacct cagaaaaagt agaaaatgga agtctatgtg |
| 1921 | atcaagaaat cgatagcatt tgcagtatag agcgtgcaga taatgacaag gaatatctag |
| 1981 | tacttacttt aacaaaaaat gatcttgaca aagcaaataa agacaaagcc aaccgatact |
| 2041 | tttctccaaa ttttaaggtg aagctgtact tcacaaaaac agtagaggag ccgtcaaatc |
| 2101 | cagaggctag cagttcaact tctgtaacac cagatgttag tgacaatgaa cctgatcatt |
| 2161 | atagatattc tgacaccact gactctgatc cagagaatga accttttgat gaagatcagc |
| 2221 | atacacaaat tacaaaagtc tgaatttttt tttatcaaga gggataaaac accatgaaaa |
| 2281 | taaacttgaa taaactgaaa atggaccttt ttttttttaa tggcaatagg acattgtgtc |
| 2341 | agattaccag ttataggaac aattctcttt tcctgaccaa tcttgtttta ccctatacat |
| 2401 | ccacagggtt ttgacacttg ttgtccagtt gaaaaaaggt tgtgtagctg tgtcatgtat |
| 2461 | ataccttttt gtgtcaaaag gacatttaaa attcaattag gattaataaa gatggcactt |
| 2521 | tcccgtttta ttccagtttt ataaaaagtg gagacagact gatgtgtata cgtaggaatt |
| 2581 | ttttcctttt gtgttctgtc accaactgaa gtggctaaag agctttgtga tatactggtt |
| 2641 | cacatcctac ccctttgcac ttgtggcaac agataagttt gcagttggct aagagaggtt |
| 2701 | tccgaagggt tttgctacat tctaatgcat gtattcgggt taggggaatg gagggaatgc |
| 2761 | tcagaaagga aataatttta tgctggactc tggaccatat accatctcca gctatttaca |
| 2821 | cacacctttc tttagcatgc tacagttatt aatctggaca ttcgaggaat tggccgctgt |
| 2881 | cactgcttgt tgtttgcgca ttttttttta aagcatattg gtgctagaaa aggcagctaa |
| 2941 | aggaagtgaa tctgtattgg ggtacaggaa tgaaccttct gcaacatctt aagatccaca |
| 3001 | aatgaaggga tataaaaata atgtcatagg taagaaacac agcaacaatg acttaaccat |
| 3061 | ataaatgtgg aggctatcaa caaagaatgg gcttgaaaca ttataaaaat tgacaatgat |
| 3121 | ttattaaata tgttttctca attgtaacga cttctccatc tcctgtgtaa tcaaggccag |
| 3181 | tgctaaaatt cagatgctgt tagtacctac atcagtcaac aacttacact tattttacta |
| 3241 | gttttcaatc ataatacctg ctgtggatgc ttcatgtgct gcctgcaagc ttcttttttc |
| 3301 | tcattaaata taaaatattt tgtaatgctg cacagaaatt ttcaatttga gattctacag |
| 3361 | taagcgtttt ttttctttga agatttatga tgcacttatt caatagctgt cagccgttcc |
| 3421 | acccttttga ccttacacat tctattacaa tgaattttgc agttttgcac attttttaaa |
| 3481 | tgtcattaac tgttagggaa ttttacttga atactgaata catataatgt ttatattaaa |
| 3541 | aaggacattt gtgttaaaaa ggaaattaga gttgcagtaa actttcaatg ctgcacacaa |
| 3601 | aaaaaagaca tttgattttt cagtagaaat tgtcctacat gtgctttatt gatttgctat |
| 3661 | tgaaagaata gggttttttt tttttttttt tttttttttt ttaaatgtgc agtgttgaat |
| 3721 | catttcttca tagtgctccc ccgagttggg actagggctt caatttcact tcttaaaaaa |
| 3781 | aatcatcata tatttgatat gcccagactg catacgattt taagcggagt acaactacta |
| 3841 | ttgtaaagct aatgtgaaga tattattaaa aaggtttttt tttccagaaa tttggtgtct |
| 3901 | tcaaattata ccttcacctt gacatttgaa tatccagcca ttttgtttct taatggtata |
| 3961 | aaattccatt ttcaataact tattggtgct gaaattgttc actagctgtg gtctgaccta |
| 4021 | gttaatttac aaatacagat tgaataggac ctactagagc agcatttata gagtttgatg |
| 4081 | gcaaatagat taggcagaac ttcatctaaa atattcttag taaataatgt tgacacgttt |
| 4141 | tccatacctt gtcagtttca ttcaacaatt tttaaatttt taacaaagct cttaggattt |
| 4201 | acacatttat atttaaacat tgatatatag agtattgatt gattgctcat aagttaaatt |
| 4261 | ggtaaagtta gagacaacta ttctaacacc tcaccattga aatttatatg ccaccttgtc |
| 4321 | tttcataaaa gctgaaaatt gttacctaaa atgaaaatca acttcatgtt ttgaagatag |
| 4381 | ttataaatat tgttctttgt tacaatttcg ggcaccgcat attaaaacgt aactttattg |
| 4441 | ttccaatatg taacatggag ggccaggtca taaataatga cattataatg ggcttttgca |
| 4501 | ctgttattat ttttcctttg gaatgtgaag gtctgaatga gggttttgat tttgaatgtt |
| 4561 | tcaatgtttt tgagaagcct tgcttacatt ttatggtgta gtcattggaa atggaaaaat |
| 4621 | ggcattatat atattatata tataaatata tattatacat actctcctta ctttatttca |
| 4681 | gttaccatcc ccatagaatt tgacaagaat tgctatgact gaaaggtttt cgagtcctaa |
| 4741 | ttaaaacttt atttatggca gtattcataa ttagcctgaa atgcattctg taggtaatct |
| 4801 | ctgagtttct ggaatatttt cttagacttt ttggatgtgc agcagcttac atgtctgaag |
| 4861 | ttacttgaag gcatcacttt taagaaagct tacagttggg ccctgtacca tcccaagtcc |
| 4921 | tttgtagctc ctcttgaaca tgtttgccat acttttaaaa gggtagttga ataaatagca |
| 4981 | tcaccattct ttgctgtggc acaggttata aacttaagtg gagtttaccg gcagcatcaa |
| 5041 | atgtttcagc tttaaaaaat aaaagtaggg tacaagttta atgtttagtt ctagaaattt |
| 5101 | tgtgcaatat gttcataacg atggctgtgg ttgccacaaa gtgcctcgtt tacctttaaa |
| 5161 | tactgttaat gtgtcatgca tgcagatgga aggggtggaa ctgtgcacta aagtgggggc |
| 5221 | tttaactgta gtatttggca gagttgcctt ctacctgcca gttcaaaagt tcaacctgtt |
| 5281 | ttcatataga atatatatac taaaaaattt cagtctgtta aacagcctta ctctgattca |
| 5341 | gcctcttcag atactcttgt gctgtgcagc agtggctctg tgtgtaaatg ctatgcactg |
| 5401 | aggatacaca aaaataccaa tatgatgtgt acaggataat gcctcatccc aatcagatgt |
| 5461 | ccatttgtta ttgtgtttgt taacaaccct ttatctctta gtgttataaa ctccacttaa |
| 5521 | aactgattaa agtctcattc ttgtcaaaaa aaaaaaaaaa aaaaaaaaaa aa |

By “Programmed cell death protein 4 (PDCD4) polypeptide” is meant a polypeptide or fragment thereof having at least 85% amino acid identity to NCBI Accession No. NP_001186421 and having tumor suppressor activity (e.g., increasing apoptosis). An exemplary PDCD4 polypeptide sequence is provided below (SEQ ID NO: 44):

| | |
|---|---|
| 1 | mdveneqiln vnpaenagte eikneingnw isassinear inakakrrlr knssrdsgrg |
| 61 | dsvsdsgsda lrsgltvpts pkgrlldrrs rsgkgrglpk kggaggkgvw gtpgqvydve |
| 121 | evdvkdpnyd ddqencvyet vvlplderaf ektltpiiqe yfehgdtnev aemlrdlnlg |
| 181 | emksgvpvla vslalegkas hremtsklls dlcgtvmstt dveksfdkll kdlpelaldt |
| 241 | prapqlygqf iaravgdgil cntyidsykg tvdcvqaraa ldkatvllsm skggkrkdsv |
| 301 | wgsgggqqsv nhlvkeidml lkeyllsgdi seaehclkel evphfhhelv yeaiimvles |
| 361 | tgestfkmil dllkslwkss titvdqmkrg yeriyneipd inldvphsys vlerfveecf |
| 421 | qagiiskqlr dlcpsrgrkr fvsegdggrl kpesy |

By “Programmed cell death protein 4 (PDCD4) nucleic acid molecule” is meant a polynucleotide encoding a PDCD4 polypeptide. An exemplary PDCD4 nucleic acid molecule is provided at NCBI Accession No. NM_001199492. An exemplary PDCD4 mRNA transcript is provided below (SEQ ID NO: 45):

| | |
|---|---|
| 1 | cttttcctcc tcagctccgg ctccgccgcc acgattggcc agccgaccac ccggcctcgg |
| 61 | ccaataagcg ccgccctctc gcccccgtgt tactgggtag aagaaaacaa aaacaaacag |
| 121 | agcgagaagg gccagagact ctccgaggcg gcggcagaga cagaagagcg gggtcggggc |
| 181 | cggctgacca ggaacctggg cgagcagcgg cgggggcccg agggattctg aaggaagatt |
| 241 | tccattaggt aatttgttta atcagtgcaa gcgaaattaa gggaaaatgg atgtagaaaa |
| 301 | tgagcagata ctgaatgtaa accctgcaga aaatgctggg actgaggaaa taaagaatga |
| 361 | aataaatgga aattggattt cagcatcctc cattaacgaa gctagaatta atgccaaggc |
| 421 | aaaaaggcga ctaaggaaaa actcatcccg ggactctggc agaggcgatt cggtcagcga |
| 481 | cagtgggagt gacgccctta gaagtggatt aactgtgcca accagtccaa agggaaggtt |
| 541 | gctggatagg cgatccagat ctgggaaagg aaggggacta ccaaagaaag gtggtgcagg |
| 601 | aggcaaaggt gtctggggta cacctggaca ggtgtatgat gtggaggagg tggatgtgaa |
| 661 | agatcctaac tatgatgatg accaggagaa ctgtgtttat gaaactgtag ttttgccttt |
| 721 | ggatgaaagg gcatttgaga agactttaac accaatcata caggaatatt ttgagcatgg |
| 781 | agatactaat gaagttgcgg aaatgttaag agatttaaat cttggtgaaa tgaaaagtgg |
| 841 | agtaccagtg ttggcagtat ccttagcatt ggaggggaag gctagtcata gagagatgac |
| 901 | atctaagctt ctttctgacc tttgtgggac agtaatgagc acaactgatg tggaaaaatc |
| 961 | atttgataaa ttgttgaaag atctacctga attagcactg gatactccta gagcaccaca |
| 1021 | gttggtgggc cagtttattg ctagagctgt tggagatgga attttatgta atacctatat |
| 1081 | tgatagttac aaaggaactg tagattgtgt gcaggctaga gctgctctgg ataaggctac |
| 1141 | cgtgcttctg agtatgtcta aaggtggaaa gcgtaaagat agtgtgtggg gctctggagg |
| 1201 | tgggcagcaa tctgtcaatc accttgttaa agagattgat atgctgctga aagaatattt |
| 1261 | actctctgga gacatatctg aagctgaaca ttgccttaag gaactggaag tacctcattt |
| 1321 | tcaccatgag cttgtatatg aagctattat aatggtttta gagtcaactg gagaaagtac |
| 1381 | atttaagatg attttggatt tattaaagtc cctttggaag tcttctacca ttactgtaga |
| 1441 | ccaaatgaaa agaggttatg agagaattta caatgaaatt ccggacatta atctggatgt |
| 1501 | cccacattca tactctgtgc tggagcggtt tgtagaagaa tgttttcagg ctggaataat |
| 1561 | ttccaaacaa ctcagagatc tttgtccttc aaggggcaga aagcgttttg taagcgaagg |
| 1621 | agatggaggt cgtcttaaac cagagagcta ctgaatataa gaactcttgc agtcttagat |
| 1681 | gttataaaaa tatatatctg aattgtaaga gttgttagca caagtttttt tttttttttt |
| 1741 | ttttaagcac ttgttttggg tacaaggcat ttctgacatt ttataaacct acatttaagg |
| 1801 | ggaattttta aaggaaatgt tttttctttt ttttttgttt ttcgaggggg caaggaggga |
| 1861 | cagaaaagta acctcttctt aagtggaata ttctaataag ctaccttttg taagtgccat |
| 1921 | gtttattatc taatcattcc aagttttgca ttgatgtctg actgccactc ctttctttca |
| 1981 | aggacagtgt tttttgtagt aaaatcactg gtttatacaa agctttattt agggggtaaa |
| 2041 | gttaagctgc taaaacccca tgttggctgc tgctgttgag atactgtgct ttgggagtaa |
| 2101 | aaaaagaaag ttatttcttt gtcttaaaga atttttaaaa aattagtcat gagacttatt |
| 2161 | catctttcca gggaacatac tgattggtct taaaagacta gacagttaag taaaaggtgg |
| 2221 | ctggaacatc tatttttcta caaaactgga aaaatgaacc tggttctaga agaatgtaca |
| 2281 | ccaaaataaa acatgtgaag cagtattgat tctttattgg gagtacattt ttttaggtct |
| 2341 | cttaaacttt aatttcacac agtaaatttt gaatctcata aggaagcata tttgaaccta |
| 2401 | gtcaatttaa tcttagtgtt cccttgaaaa ctttttttcc ctacaaaatt ttaagtgaaa |
| 2461 | aatacaatag taaattaaga ttacactggg gaaaaaaatg caggtatcac tttactccat |
| 2521 | tgttatctga cctagagctt aattaagttt tagaaatatg taataccttc catcattcca |
| 2581 | tcatccttaa attctgttac caaataatgg ctaatgttac aaaaagttat actccagaga |
| 2641 | cccaaagctt gacatttacc taatgtatga gaaaatatta ccaattaaca ataaagaatg |
| 2701 | atcatatttt taacctcttt tacatagcct aataactcag caaggcctca acgtctgtgc |
| 2761 | taatttaaac tgccaaatat tgactgcagc aaacaagaat tatattcaga atttatgagg |
| 2821 | gtactgttag gagtatactg cttacaggtt tagatatagt ctgttagaat taaaaccaag |
| 2881 | tttagtgttc atatttacct catgggcttt atcaagccca tattacctca gcttatatat |
| 2941 | agttaccatt tttaggtttt taattgtttg acacttggat gataaatgca gtcattttat |
| 3001 | tctcaagtgc ttaaaattaa tgtaattaaa agcttagctg actacagaat aggtgagggt |
| 3061 | ttcttaaaaa tgagatttaa gggctgggca cggtggctca tgcctgtaat cccagcactt |
| 3121 | tgggaggccg aggtgggcgg atcacttgag gttgggagtt catgaccagc ttgaccaaca |
| 3181 | tgaagaaacc ctgtctctat taaaaataca aaagtagcca ggcatggtgg cgcatgcgtg |
| 3241 | taatcccagc tacttgggag gctgaggcag gagaattgct tgaacctggg aggcagaggt |
| 3301 | tgcagtgagt cgagatggtg ccattgctct cgtttgggca acaagagtga aactcttgtc |
| 3361 | tcaaaaaaaa aaaaaaatga ggtttaagac agttttgtca ttactggtgg gatctggtca |
| 3421 | cacaagatag cattaaacgt gacatggcac ataaaattgg ttaaaaaatt ttgtttttta |
| 3481 | attacgtaat gtaaaagccc aacaaacact ttatgcaaga ttggaatgta tcttcaaatt |
| 3541 | cagatttaat aaacatgtaa agatcctctg taaaaaaaaa aaaaaaaaaa aaaaaaaaaa |
| 3601 | aa |

By “neoplasm” is meant a disease or disorder characterized by excess proliferation or reduced apoptosis. Illustrative neoplasms for which the invention can be used include, but are not limited to breast cancer, leukemias (e.g., acute leukemia, acute lymphocytic leukemia, acute myelocytic leukemia, acute myeloblasts leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangio sarcoma, lymphangioendotheliosarcoma, synovioma, mesothelioma, Ewing's tumor, leiomyosarcoma, rhabdomyosarcoma, colon carcinoma, ovarian cancer, pancreatic cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid. Examples of such analogs include, without limitation, phosphorothioates, phosphoramidates, boranophosphates, methylphosphonates, 2-O-alkyl ribonucleotides, and peptide nucleic acids (PNAs).

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, e.g., Usman and McSwiggen, supra; Eckstein, et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-methoxyethoxy, 2′-fluoro, 2′-thio, 2′-allyl, 2′-O-[2-(methylamino)-2-oxoethyl], 4′-thio, 4′-CH₂—O-2′-bridge, 4′-(CH₂)₂—O-2′-bridge, 2′-LNA, and 2′-O—(N-methylcarbamate) or those comprising base analogs. In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878.

By “disease” is meant any condition or disorder that damages or interferes with the normal function of a cell, tissue, or organ. Examples of diseases include cancer, pulmonary arterial hypertension, arthritis, cirrhosis, diabetes, or heart disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “complementary sequence” or “complement” is meant a nucleic acid base sequence that can form a double-stranded structure by matching base pairs to another polynucleotide sequence. Base pairing occurs through the formation of hydrogen bonds, which may be Watson-Crick, Hoogsteen or reversed Hoogsteen hydrogen bonding, between complementary nucleobases. For example, adenine and thymine are complementary nucleobases that pair through the formation of hydrogen bonds. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary, respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being basepaired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

As used herein, an “antisense” oligonucleotide or polynucleotide is a nucleic acid molecule having a nucleic acid sequence that is substantially complementary to a target polynucleotide or a portion thereof and has the ability to specifically hybridize to the target polynucleotide.

As used herein, “guide strand” refers to a single stranded nucleic acid molecule of an miRNA, which has a sequence sufficiently complementary to that of a target mRNA to hybridize to the target mRNA (e.g., in the 5′ UTR, the coding region, or the 3′ UTR) and to decrease or inhibit its translation. A guide strand is also termed an “antisense strand.”

As used herein, “target RNA” refers to an RNA that is subject to modulation guided by an inhibitory nucleic acid or portion thereof (e.g., an antisense polynucleotide or a strand of an miRNA), such as targeted cleavage or steric blockage. The target RNA could be, for example genomic viral RNA, mRNA, a pre-mRNA, or a non-coding RNA. In various embodiments, the preferred target is miRNA, such as miRNA involved in cancer, such as miR-17 or miR-21. In certain embodiments, an inhibitory nucleic acid molecule targets an miRNA, but does not target or does not substantially target an mRNA that is targeted by the miRNA.

As used herein, “seed region” refers to the portion of an oligonucleotide strand that hybridizes to a target RNA (e.g. an miRNA), and involves a sequence that is complementary or substantially complementary to the target RNA. A seed region may be 6, 7, or 8 nucleotides in length.

As used herein, “passenger strand” refers to an oligonucleotide strand of an miRNA, which has a sequence that is complementary or substantially complementary to that of the guide strand. In some embodiments, the passenger strand may target an mRNA by hybridizing to the target mRNA (e.g., in the 5′ UTR, the coding region, or the 3′ UTR) and to decrease or inhibit its translation A passenger strand is also termed a “sense strand.”

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative amino acid substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e^{″3} and e^{″100} indicating a closely related sequence.

Nucleic acid molecules useful in the methods of the invention include any nucleic acid molecule that regulates or encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). In one embodiment, therapeutic oligonucleotides hybridize in physiological buffer at 37° C. in patients.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS.

Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the biomarker to be detected.

The phrase “differentially present” refers to differences in the quantity and/or the frequency of a biomarker present in a sample taken from subjects having a disease as compared to a control subject. A biomarker can be differentially present in terms of quantity, frequency or both. A polypeptide or polynucleotide is differentially present between two samples if the amount of the polypeptide or polynucleotide in one sample is statistically significantly different from the amount of the polypeptide or polynucleotide in the other sample, such as a reference. Alternatively or additionally, a polypeptide or polynucleotide is differentially present between two sets of samples if the frequency of detecting the polypeptide or polynucleotide in a diseased subjects' samples is statistically significantly higher or lower than in the control samples. A biomarker that is present in one sample, but undetectable in another sample is differentially present.

By “effective amount” is meant the amount of an agent or compound required to reduce or improve at least one symptom of a disease relative to an untreated patient. The effective amount of active compound(s) used to practice the present invention for therapeutic treatment of a disease varies depending upon the manner of administration, the age, body mass, and general health of the subject.

The term “expression” as used herein is defined as the transcription and/or translation of a particular nucleotide sequence driven by its promoter.

By “fragment” is meant a portion of a polynucleotide or nucleic acid molecule. This portion contains, preferably, at least 10%, 20%, 30%, 40%, 50%, 60%, 70%, 80%, or 90% of the entire length of the reference nucleic acids. A fragment may contain 10, 20, 30, 40, 50, 60, 70, 80, 90, or 100, 200, 300, 400, 500, 600, 700, 800, 900, 1000, 1500, 2000 or 2500 (and any integer value in between) nucleotides. The fragment, as applied to a nucleic acid molecule, refers to a subsequence of a larger nucleic acid. A “fragment” of a nucleic acid molecule may be at least about 10 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

As used herein, the term “inhibit” is meant to refer to a decrease in biological state. For example, the term “inhibit” may be construed to refer to the ability to negatively regulate the expression, stability or activity of a protein, including but not limited to transcription of a protein mRNA, stability of a protein mRNA, translation of a protein mRNA, stability of a protein polypeptide, a protein post-translational modifications, a protein activity, a protein signaling pathway or any combination thereof.

Further, the term “inhibit” may be construed to refer to the ability to negatively regulate the expression, stability or activity of a miRNA, wherein such inhibition of the miRNA may affect modulation of a gene, protein mRNA, stability of a protein mRNA, translation of a protein mRNA, stability of a protein, a protein post-translational modifications, and/or a protein activity.

“Instructional material,” as that term is used herein, includes a publication, a recording, a diagram, or any other medium of expression that may be used to communicate the usefulness of the compounds of the invention. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds of the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which may be hydrolyzed into the monomeric “nucleotides.” The monomeric nucleotides may be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means. The following abbreviations for the commonly occurring nucleic acid bases are used. “A” refers to adenine, “C” refers to cytosine, “G” refers to guanine, “T” refers to thymine, and “U” refers to uracil. The term “RNA” as used herein is defined as ribonucleic acid. The term “recombinant DNA” as used herein is defined as DNA produced by joining pieces of DNA from different sources.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

As used herein, the terms “prevent,” “preventing,” “prevention,” and the like refer to reducing the probability of developing a disorder or condition in a subject, who does not have, but is at risk of or susceptible to developing a disorder or condition.

By “reduces” or “decreases” is meant a negative alteration of at least 10%, 25%, 50%, 75%, or 100%.

By “reference” is meant a standard or control. A “reference” is also a defined standard or control used as a basis for comparison.

As used herein, “sample” or “biological sample” refers to anything, which may contain the biomarker (e.g., polypeptide, polynucleotide, or fragment thereof) for which a biomarker assay is desired. The sample may be a biological sample, such as a biological fluid or a biological tissue. In one embodiment, a biological sample is a tissue sample including pulmonary arterial endothelial cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the percentage of biomarker-detected subjects with a particular disease.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, the terms “treat,” treating,” “treatment,” “therapy,” and the like refer to reducing or improving a disorder and/or symptom associated therewith. It will be appreciated that, although not precluded, treating a disorder or condition does not require that the disorder, condition or symptoms associated therewith be completely ameliorated or eliminated.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Ranges provided herein are understood to be shorthand for all of the values within the range. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

miRNA Inhibitors

The present invention provides nucleic acids that inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

According to the current view (Tijsterman and Plasterk, 2004, Cell 117(1) 1-3), after cutting by Dicer, the guide strand is retained in the RNA-induced silencing complex (RISC) to perform the inhibitory function towards its target mRNAs, while the passenger strand dissociates and is degraded. However, the applicants have observed that the passenger strand can also occasionally act as a functional miRNA. In that case, there is a competition between the guide strand and its passenger strand when both of them target the same mRNA. As an unintended result, an antisense oligonucleotide against a miRNA that has a functional passenger strand can act itself as a mimic of the miRNA passenger strand. This situation requires the antisense oligonucleotide to include a majority of the miRNA passenger strand sequence.

As described in the results herein, if the passenger strand has more binding sites in the target mRNA than the guide strand, the passenger strand could win the competition with the original miRNA. Thus, antisense oligonucleotide inhibition of the original miRNA might decrease, rather than increase, target mRNA translation, as an unintended consequence. Applicants have discovered that designing an antisense oligonucleotide that only hybridizes to about half of the mature guide miRNA, that is homologous to the 3′ region but not the 5′ seed region of the passenger strand, which is required for inhibitory function of miRNAs, overcomes the inhibitory function of passenger strand mimics.

With these sequence restrictions, the antisense oligonucleotide can successfully inhibit the guide strand, while not acting as a passenger strand mimic. This is in contrast to previous efforts to reduce off-target specificity using chemically modified antisense oligonucleotides. Indeed, the current standard technology provides miRNA inhibitors without considering the possibility of creating mimics of the passenger strand. Such products have the potential to introduce non-specific effects to the miRNA under study by promoting functionality of its passenger strand, especially when both of them target the same mRNAs, with more available binding sites for the passenger strand. Thus, the resulting biological observation may be contradictory to what is expected when the miRNA in interest is inhibited specifically.

The present invention features short nucleic acid molecules that function as miRNA inhibitors in a cell. The term “short” refers to a length of a single polynucleotide that is 10, 11, 12, 13, 14, 15, 16, 17, 18, 19, 20, 21, 22, 23, 24, 25, 50, 100, or 150 nucleotides or fewer, including all integers or ranges derivable there between. In various embodiments, miRNA inhibitor is between about 10 to 25 nucleotides in length and comprises a 5′ to 3′ sequence (e.g., a seed region) that is at least 90% complementary to the 5′ to 3′ sequence of a mature miRNA. In certain embodiments, an miRNA inhibitor molecule is 10, 11, 12, 13, 14, 15, 16, 17, 18, 19, 20, 21, 22, 23, 24, or 25 nucleotides in length, or any range derivable therein. Moreover, an miRNA inhibitor may have a sequence (from 5′ to 3′) that is or is at least 50, 60, 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for a miRNA inhibitor. Moreover, that portion of the nucleic acid sequence can be altered so that it still comprises the appropriate percentage of complementarily to the sequence of a mature miRNA. In particular embodiments, an miR-17 inhibitory nucleic acid includes the nucleic acid sequence 5′-GTAAGCACTTTG-3′(SEQ ID NO: 1) and binds miR-17-5p. In other embodiments, an miR-21 inhibitory nucleic acid includes the nucleic acid sequence 5′-TCTGATAAGCTA-3′(SEQ ID NO: 2) and binds miR-21-5p.

An important consideration for the efficacy of nucleic acid molecules of the invention is degradation by nucleases. Examples of modifications contemplated for the phosphate backbone include boranophosphate, methylphosphonate, phosphorothioate, and phosphotriester modifications such as alkylphosphotriesters, and the like. In the nucleic acid molecules of the invention, phosphorothioate, methylphosphonate, or boranophosphate modifications directly stabilize the internucleoside phosphate linkage. Boranophosphate modified RNAs are highly nuclease resistant, potent as silencing agents, and are relatively non-toxic. Boranophosphate DNAs are synthesized by an H-phosphonate route (U.S. Pat. No. 5,859,231). Boranophosphate modified RNAs cannot be manufactured using standard chemical synthesis methods and instead are made by in vitro transcription (IVT) (Hall et al., 2004 and Hall et al., 2006). Phosphorothioate and methylphosphonate modifications can be readily placed in a nucleic acid molecule of the invention at any desired position and can be made using standard chemical synthesis methods.

A variety of substitutions can be placed at the 2′-position of the ribose. Such 2′ modifications generally increase duplex stability (Tm) and can greatly improve nuclease resistance. Examples of modifications contemplated for the sugar moiety include 2′-O-alkyl, such as 2′-O-methyl, 2′-fluoro, 2′-amino modifications and the like (see, e.g., Amarzguioui et al., 2003). Examples of modifications contemplated for the base groups include abasic sugars, modified pyrimidines, modified purines, and the like.

Locked nucleic acids (LNAs) are a particular class of 2′-modification that can be incorporated to stabilize nucleic acid molecules of the invention. Many other modifications are known and can be used so long as the above criteria are satisfied. Examples of modifications are also disclosed in U.S. Pat. Nos. 5,684,143, 5,858,988 and 6,291,438 and in U.S. published patent application No. 2004/0203145 A1. Other modifications are disclosed in Herdewijn (2000), Eckstein (2000), Rusckowski et al. (2000), Stein et al. (2001); Vorobjev et al. (2001).

Peptide nucleic acids (PNAs) are chemically synthesized oligoamides of N-aminoethyl glycine with nucleic acid bases attached to the alpha amine of glycine (Nielsen, P. E., et al., 1991, Science 254(5037) 1497-1500, U.S. Pat. No. 5,539,082).

### Triple Negative Breast Cancer

The oncomiR miR-17-5p, which inhibits translation of tumor suppressors PTEN and PDCD4, and miR-21-5p, which also inhibits PTEN translation, are typically overexpressed in TNBC cells (Farazi et al., 2011, Cancer Research 71(13):4443-4453). Without being bound to a particular theory, it is hypothesized that knockdown or blockade of elevated miR-17-5p or miR-21-5p by systemic anti-miRNA agents specifically targeting triple negative breast cancer (TNBC) cells would offer a novel therapy for disseminated drug-resistant TNBC, restoring the balance of homeostasis in TNBC cells by re-differentiating them to a normal phenotype. However, some activity of passenger strands has been asserted (Mah et al., 2010, Crit Rev Eukaryot Gene Expr 20(2):141-8). Without being bound to a particular theory, the results described herein indicate that the miR-17-3p passenger strand targets PTEN and PDCD4 mRNAs.

### Therapeutic Methods

In one embodiment, the present invention provides a method of treating diseases, including neoplasia (e.g., breast cancer). Advantageously, the invention provides a method for treating diseases, including neoplasia (e.g., breast cancer) that are less susceptible to conventional treatment methods. The method involves administering to a subject having a neoplasm an effective amount of one or more polynucleotide inhibitors of miR-17 and/or miR-21. Preferably, such an agent is administered as part of a composition additionally comprising a pharmaceutically acceptable carrier. Preferably this method is employed to treat a subject suffering from or susceptible to a neoplasm. Other embodiments include any of the methods described herein wherein the subject is identified as in need of the indicated treatment. Another aspect of the invention is the manufacture of a medicament for treating a neoplasm (e.g., breast tumor) in a subject. Preferably, the medicament is used for treatment or prevention in a subject of a disease, disorder or symptom set forth herein.

Examples of cancer that can be treated according to the methods of the present invention include but are not limited to, carcinoma, lymphoma, blastoma, sarcoma, and leukemia or lymphoid malignancies. More particular examples of such cancers include kidney or renal cancer, breast cancer, colon cancer, rectal cancer, colorectal cancer, lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, liver cancer, bladder cancer, cancer of the peritoneum, hepatocellular cancer, gastric or stomach cancer including gastrointestinal cancer, gastrointestinal stromal tumors (GIST), pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulvar cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, urinary tract carcinomas, thyroid carcinomas, Wilm's tumor, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia, chronic myeloblastic leukemia; and post-transplant lymphoproliferative disorder (PTLD), as well as abnormal vascular proliferation associated with phakomatoses, edema (such as that associated with brain tumors), and Meigs' syndrome. “Tumor”, as used herein, refers to all neoplastic cell growth and proliferation, whether malignant or benign, and all pre-cancerous and cancerous cells and tissues.

In the context of treatment for cancer, the polynucleotide inhibitors of the present invention can optionally be administered to a patient in combination with other chemotherapeutic agents. Suitable chemotherapeutic agents include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins, capecitabine; and pharmaceutically acceptable salts, acids or derivatives of any of the above. Additional information on the methods of cancer treatment is provided in U.S. Pat. No. 7,285,522, incorporated by reference in its entirety.

### Pharmaceutical Compositions and Formulations

The invention also encompasses the use of a pharmaceutical composition of the invention to practice the methods of the invention. Such a pharmaceutical composition may be provided in a form suitable for administration to a subject, and may be comprise one or more pharmaceutically acceptable carriers, one or more additional ingredients, or some combination of these. The at least one composition of the invention may comprise a physiologically acceptable salt, such as a compound contemplated within the invention in combination with a physiologically acceptable cation or anion, as is well known in the art.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In one embodiment, the compositions of the invention are formulated using one or more pharmaceutically acceptable excipients or carriers. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

### Polynucleotide Delivery

Nucleic acid molecules encoding polynucleotides of the invention can be delivered to cells (e.g., neoplastic cells, tumor cells). The nucleic acid molecules must be delivered to the cells of a subject in a form in which they can be taken up so that therapeutically effective levels of a polynucleotide of the invention can be produced. Transducing viral (e.g., retroviral, adenoviral, and adeno-associated viral) vectors can be used, especially because of their high efficiency of infection and stable integration and expression (see, e.g., Cayouette et al., 1997, Human Gene Therapy 8:423-430; Kido et al, 1996, Current Eye Research 15:833-844; Bloomer et al., 1997, Journal of Virology 71:6641-6649; Naldini et al., 1996, Science 272:263-267; and Miyoshi et al., 1997, Proc. Natl. Acad. Sci. U.S.A. 94:10319). For example, a polynucleotide can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an expression vector of the invention to a target cell, tumor tissue, or systemically. A nucleic acid molecule can also be introduced into a cell by administering the nucleic acid molecule in the presence of lipofectin (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by microinjection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. In other embodiments, inhibitory nucleic acids of the invention can be delivered without transfection or electroporation. For example, inhibitory miRNA can be covalently linked to a D(CSKC) tetrapeptide analog of insulin-like growth factor 1 (IGF1) at the C-terminus of PNA to direct endocytosis into cells that overexpress IGF1R (Basu and Wickstrom, 1997, Bioconjugate Chemistry 8(4):481-488).

Expression of a reporter construct of the invention can be directed from any suitable promoter and regulated by any appropriate mammalian regulatory element. Alternatively, regulation can be mediated by cognate regulatory sequences or, if desired, by regulatory sequences derived from a heterologous source, including any of the promoters or regulatory elements described above.

Desirably, the cells and cell lines disclosed herein are engineered to express an expression vectors described herein. Typically, an expression vector is used to transfect the cells. The term “transfection” as used herein means an introduction of a foreign DNA or RNA into a cell by mechanical inoculation, electroporation, infection, particle bombardment, microinjection, or by other known methods. Alternatively, one or a combination of expression vectors can be used to transform the cells and cell lines. The term “transformation” as used herein means a stable incorporation of a foreign DNA or RNA into the cell which results in a permanent, heritable alteration in the cell. A variety of suitable methods are known in the field and have been described. See e.g., Ausubel et al., supra; Sambrook, supra; and the Promega Technical Manual.

In particular invention embodiments, a cell or cell line of choice is manipulated so as to be stably transformed by an expression vector of the invention. However, for some invention embodiments, transient expression of the vector (e.g., for less than about a week, such as one or two days) will be more helpful. Cells and cell lines that are transiently transfected or stably transformed by one or more expression vectors disclosed herein will sometimes be referred to as “recombinant”. By the phrase “recombinant” is meant that the techniques used for making cell or cell line include those generally associated with making and using recombinant nucleic acids (e.g., electroporation, lipofection, use of restriction enzymes, ligases, etc.).

In brief summary, the expression of natural or synthetic nucleic acids of the invention is typically achieved by operably linking a nucleic acid encoding the desired sequence or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., MOLECULAR CLONING: A LABORATORY MANUAL, volumes 1-3 (3^{rd} ed., Cold Spring Harbor Press, N Y 2001), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a polynucleotide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., MOLECULAR CLONING: A LABORATORY MANUAL volumes 1-3 (3^{rd} ed., Cold Spring Harbor Press, N Y 2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Administration/Dosing

In the clinical settings, delivery systems for the therapeutic composition can be introduced into a patient by any of a number of methods, each of which is familiar in the art. For instance, a pharmaceutical composition can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen, et al. PNAS 91: 3054-3057 (1994)). The preparation may also be provided to cells ex vivo. Cells containing the miRNAs (e.g., miR-424 and/or miR-503) are then administered to the patient.

The regimen of administration may affect what constitutes an effective amount. The therapeutic formulations may be administered to the patient either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or condition in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body mass/per day. One of ordinary skill in the art would be able to study the relevant factors and make the determination regarding the effective amount of the therapeutic compound without undue experimentation.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/kgKg body mass to about 5000 mg compound/kg body mass; or from about 5 mg/kg body mass to about 4000 mg/kg body mass or from about 10 mg/kg body mass to about 3000 mg/kg body mass; or from about 50 mg/kg body mass to about 2000 mg/kg body mass; or from about 100 mg/kg body mass to about 1000 mg/kg body mass; or from about 150 mg/kg body mass to about 500 mg/kg body mass. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/kg body mass. In other embodiments, it is envisaged that doses may be in the range of about 5 mg compound/kg body to about 20 mg compound/kg body mass. In other embodiments the doses may be about 8, 10, 12, 14, 16 or 18 mg/kg body mass. Of course, this dosage amount may be adjusted upward or downward, as is routinely done in such treatment protocols, depending on the results of the initial clinical trials and the needs of a particular patient.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Kits and Pharmaceutical Systems

The present compositions may be assembled into kits or pharmaceutical systems for use in ameliorating a neoplasm (e.g., breast cancer). Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention. Kits of the invention include an oligonucleotide inhibitor that prevents or decreases binding of an miRNA and its target nucleic acid molecule(s) (e.g., miR-17-5p or miR-17-3p binding to a PTEN or PDCD4 mRNA). The kit may include instructions for administering one or more inhibitory nucleic acids that bind an miRNA for the treatment of a neoplasm (e.g., triple negative breast cancer). Methods for measuring the efficacy of an agent are known in the art (e.g., measuring the IC₅₀).

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

These methods described herein are by no means all-inclusive, and further methods to suit the specific application will be apparent to the ordinary skilled artisan. Moreover, the effective amount of the compositions can be further approximated through analogy to compounds known to exert the desired effect.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### LNA Knockdown of miRNAs

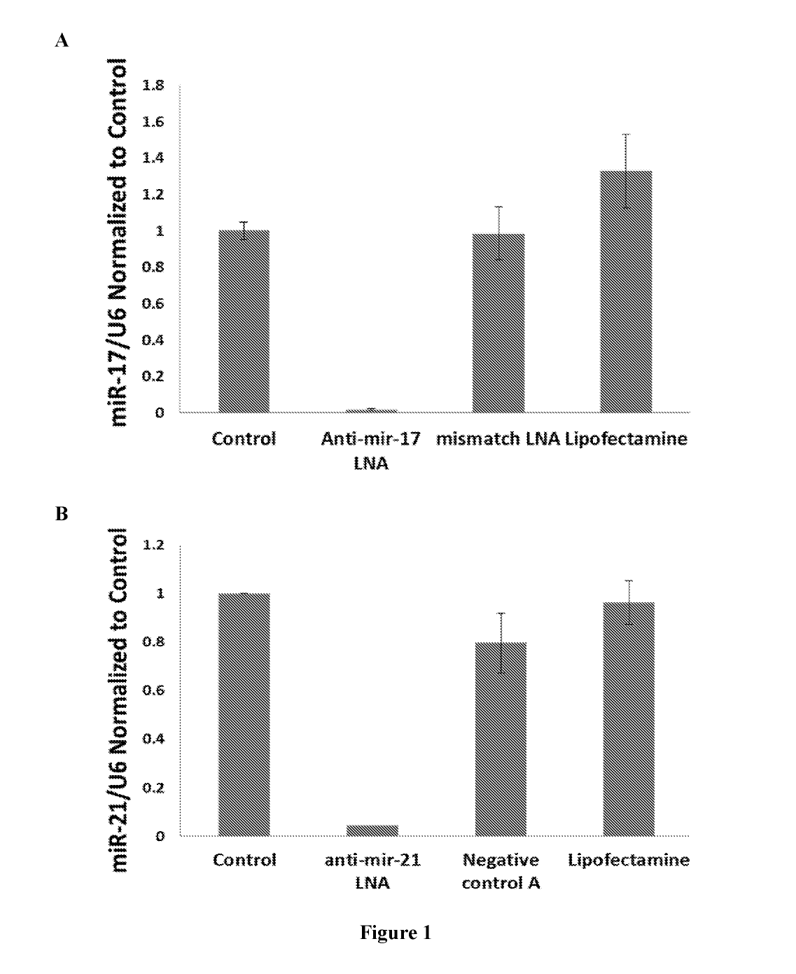

Purified 20-mer anti-miR-17-5p and anti-miR-21-5p LNA-DNA-LNA gapmers were purchased from Exiqon (Copenhagen, Denmark). qPCR showed 98% knockdown of miR-1′7-5p and miR-21-5p in MDA-MB-231 cells treated with 50 nM LNAs (FIG. 1).

### Example 2

### Bioinformatic Searches for miR-17-5p and miR-21-5p Target Sites

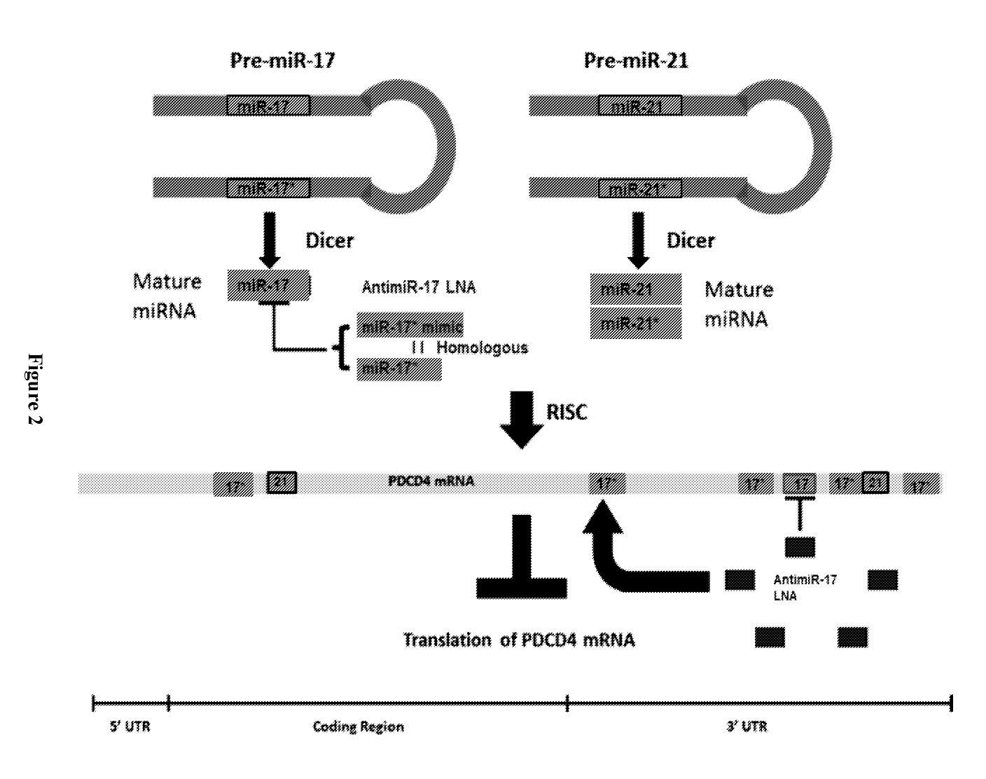

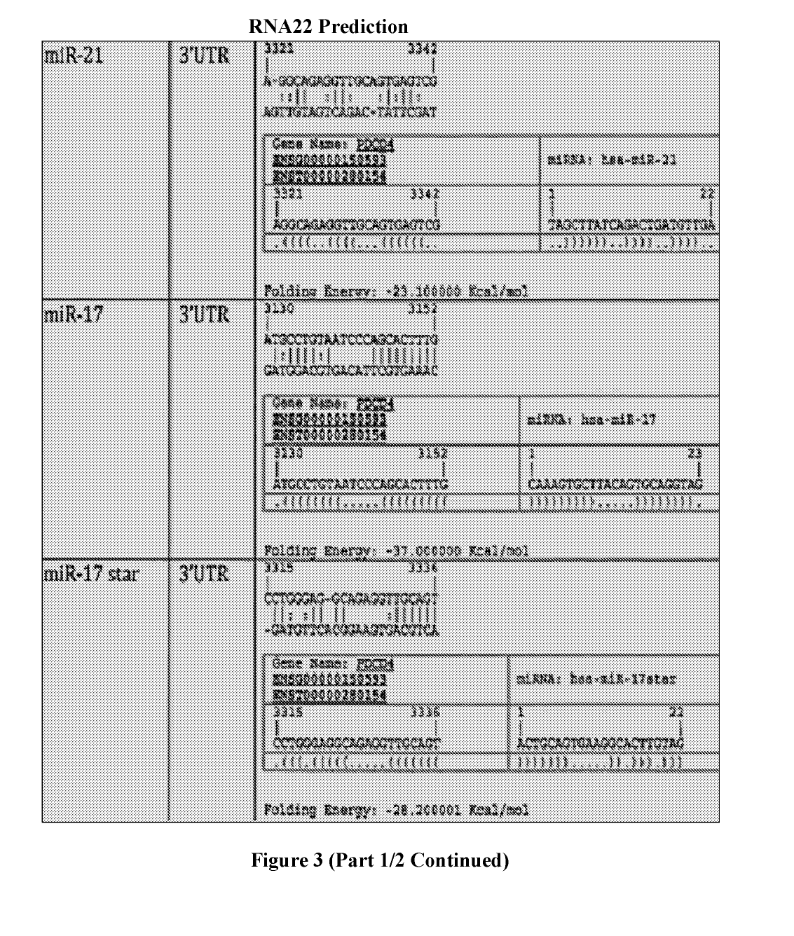

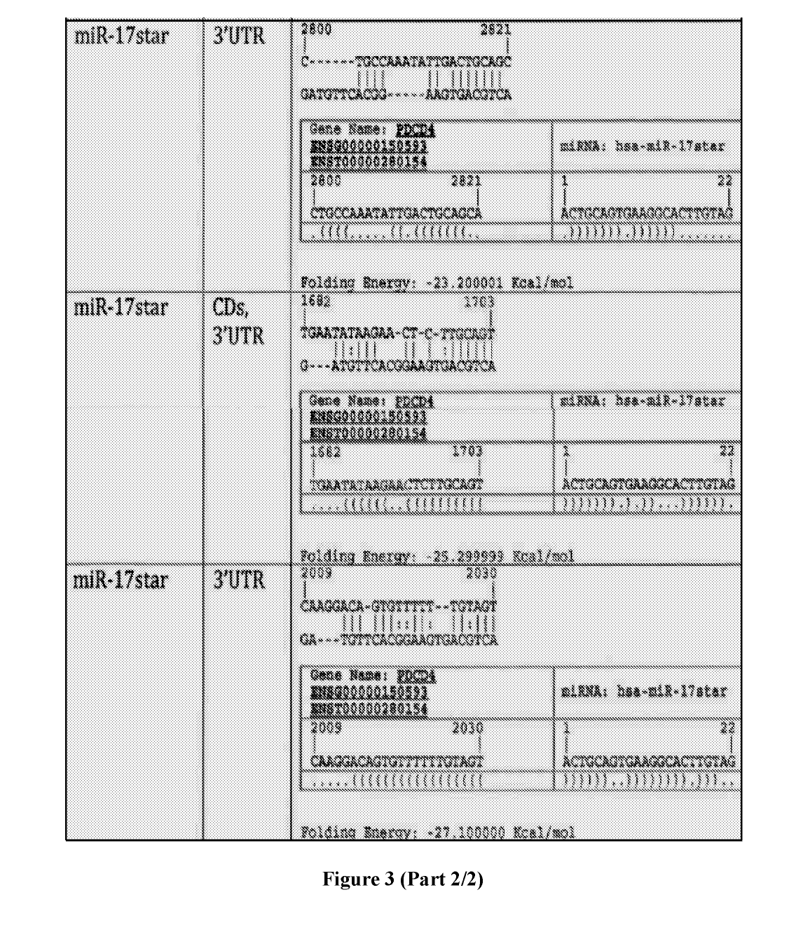

In silico searches for potential miR-17-5p and miR-21-5p target sites in the 3′UTR of PDCD4 mRNAs revealed occult miR-17-3p passenger strand targets in PTEN and PDCD4 mRNA (FIGS. 2, 3A, and 3B). Thus, an anti-miR-17-5p LNA might mimic miR-17-3p, capable of attacking novel sites in PTEN and PDCD4 mRNA. Conversely, no miR-21-3p passenger strand targets were found in PTEN or PDCD4 mRNA.

### Example 3

### Effects of miRNA Knockdown on PTEN and PDCD4 Proteins

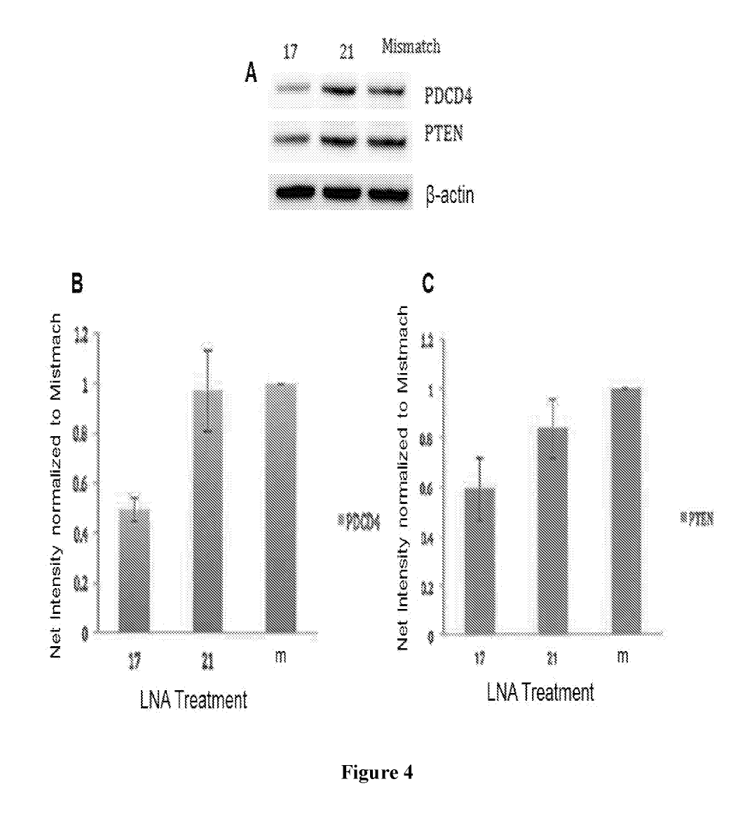

Knocking down miR-17-5p in MDA-MB-231 TNBC cells with transfected antisense locked nucleic acid (LNA) paradoxically reduced PTEN and PDCD4 protein levels (FIGS. 4A-4C). MDA-MB-231 cells were seeded in 6-well plates in L-15 medium plus 10% FBS without antibiotics the day before transfection. LNAs (50 nM final concentration) were transfected via lipofectamine 2000 in Opti-MEM for 6 hours. The medium was replaced with L-15 medium plus 10% FBS without antibiotics at the end of transfection. Total protein was extracted 48 hours post-transfection. Western blots were quantified using the Kodak Imaging Station 2000R.

Knocking down miR-17-5p in MDA-MB-231 TNBC cells with transfected antisense locked nucleic acid (LNA) paradoxically reduced PTEN and PDCD4 protein levels (FIGS. 4A-4C). Without being bound to a particular theory, it is hypothesized that the anti-miR-17-5p LNA inadvertently attacked the miR-17-3p passenger strand targets in PTEN and PDCD4 mRNA, consistent with the sequence searches. However, anti-miR-21-5p LNA did not alter PDCD4 mRNA translation in MDA-MB-231 TNBC cells.

There were more predicted binding sites for miR-17-3p passenger strand compared to miR-17-5p guide strand in the 3′UTR of PDCD4 and PTEN mRNAs (FIG. 2). Without being bound to a particular theory, whichever strand has more binding sites in the 3′UTR of a particular target mRNA, compared to the other, plays a more important role in regulating the translation of that mRNA. Thus, anti-miR-17 LNA 20-mers can mimic miR-17-3p passenger strand and further inhibit the translation of PDCD4 and PTEN mRNAs.

The miR-21-3p passenger strand had no predicted binding sites on the 3′UTR of PDCD4 or PTEN mRNAs. Therefore, anti-miR-21 LNA 20-mers that mimic the miR-21-3p passenger strand do not have inhibitory function towards mRNAs of PDCD4 and PTEN.

In a related experiment, the protein expression level of PDCD4 and PTEN was determined after treating MDA-MB-231 cells with miR-17 and miR-21 inhibitory 12-mer PNA-peptides (see Table 1).

TABLE 1
Anti-miR PNA-IGF1 tetrapeptide design

| Anti-miR PNA-IGF1 tetrapeptide | PNA Sequences and controls |
|---|---|
| Anti-miR-21 PNA-IGF1 peptide | N-TCTGATAAGCTA-AEEA-D(Cys-Ser-Lys-Cys) |
| | Where N-TCTGATAAGCTA (SEQ ID NO: 6) and |
| | AEEA-D(Cys-Ser-Lys-Cys) (SEQ ID NO: 99) |
| Anti-miR-17-5p PNA-IGF1 peptide | N-GTAAGCACTTTG-AEEA-D(Cys-Ser-Lys-Cys) |
| | Where N-GTAAGCACTTTG (SEQ ID NO: 47) and |
| | AEEA-D(Cys-Ser-Lys-Cys) (SEQ ID NO: 99) |
| Mismatch PNA-IGF1 peptide | N-TCAGATATGCTA-AEEA-D(Cys-Ser-Lys-Cys) |
| | Where N-TCAGATATGCTA (SEQ ID NO: 48) and |
| | AEEA-D(Cys-Ser-Lys-Cys) (SEQ ID NO: 99) |

MDA-MB-231 cells were seeded in 6-well plates in L-15 medium plus 10% FBS without antibiotics the day before transfection. Cells were incubated with 1 μM final concentration of PNA-peptides in L-15 medium plus 10% FBS without antibiotics for 48 hours. Total protein was extracted at the end of 48-hour incubation. Western blots were quantified using the Kodak Imaging Station 2000R.

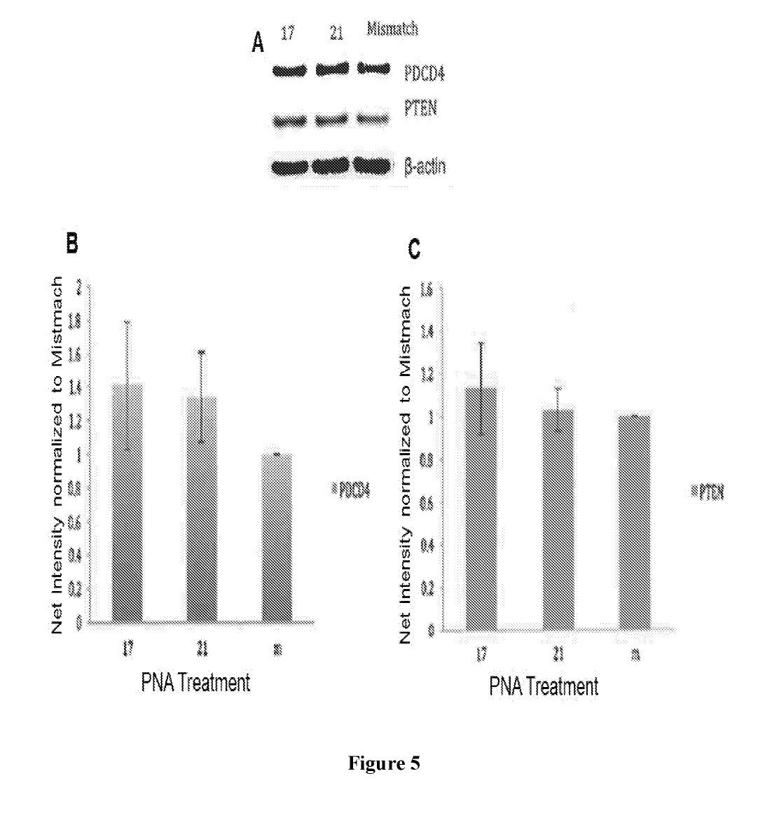

Consistent with the above results using LNAs, the protein expression level of PDCD4 was increased compared to mismatch control after treating MDA-MB-231 cells with miR-17 and miR-21 inhibitory 12-mer PNA-peptides. Protein expression level of PTEN was unchanged compared to mismatch control, consistent with the prediction program (rna22) which did not predict any potential binding sites for guide miR-17 and miR-21 on the 3′UTR of PTEN mRNA (FIGS. 5A-5C).

### Example 4

### MicroRNA:3′UTR Structures

Without being bound to a particular theory, it was hypothesized that miRNA passenger strand activity can be predicted. Each microRNA (miRNA) can target many different genes through their messenger RNAs (mRNAs) (Bartel, 2004, Cell 116(2):281-97). Antisense blocking of miRNAs has yielded inconsistent results (Hausser and Zavolan, 2014, Nat Rev Genet 15(9):599-612). Only one of the two strands in a pre-miRNA duplex is said to be selected by Ago enzyme as the mature miRNA guide strand, including a key seed sequence, while the complementary passenger strand is said to be discarded (Ha and Kim, 2014, Nat Rev Mol Cell Biol 15(8):509-24).

Tumor suppressor proteins such as phosphatase and tensin homologue (PTEN) (Depowski et al., 2001, Mod Pathol 14(7):672-676) and programmed cell death 4 (PDCD4) (Frankel et al., 2008, J Biol Chem 283(2):1026-33) are reduced in transformed cells. miR-17-5p (Yu et al., 2008, J. Cell Biol. 182(3):509-517) and miR-21-5p (Lu et al., 2008, Oncogene 27(31):4373-9) are typically overexpressed in transformed cells (Farazi et al., 2011, Cancer Research 71(13):4443-4453). miR-17-5p (Shan et al., J Cell Sci 126(Pt 6):1517-30) and miR-21-5p (Meng et al., 2007, Gastroenterology 133(2):647-58) inhibit the translation of PTEN mRNA. miR-21-5p inhibits the translation of PDCD4 mRNA (Frankel et al., 2008, J Biol Chem 283(2):1026-33). Yet passenger strands such as miR-17-3p and miR-21-3p were dismissed as nonfunctional junk RNA to be degraded, until recently (Jin et al., 2015, PLoS One 10; Mah et al., 2010, Crit Rev Eukaryot Gene Expr 20(2):141-8).

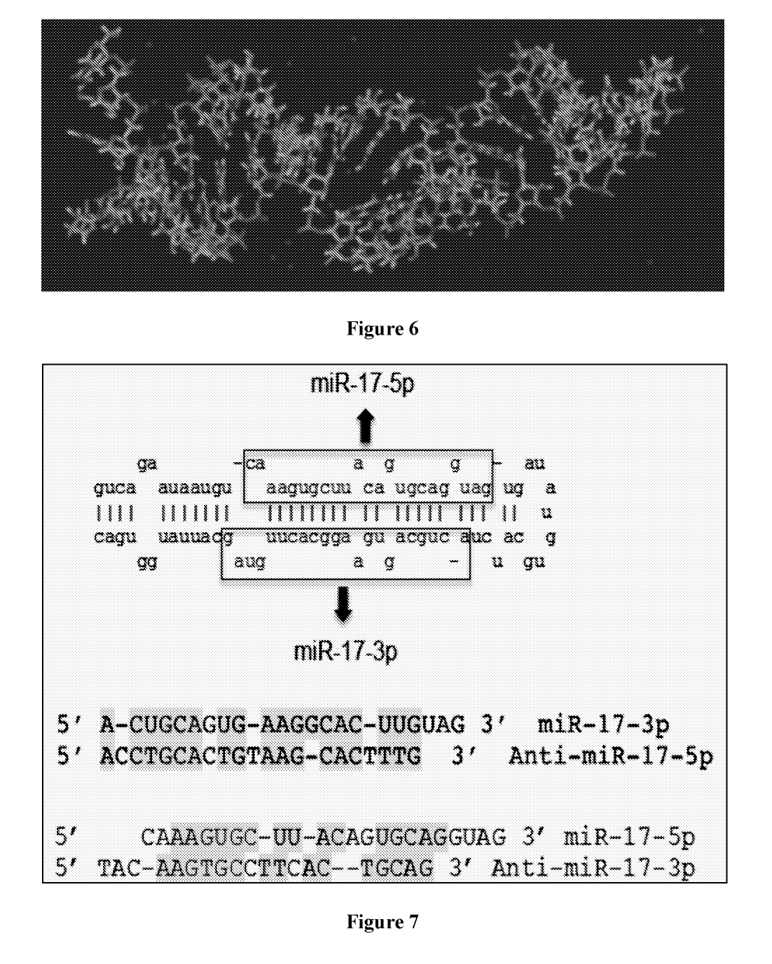

In light of conventional wisdom, it was unexpected and surprising to find that knocking down miR-17-5p in human MDA-MB-231 cells with transfected DNA-LNA chimeras paradoxically reduced PTEN and PDCD4 mRNA translation (Jin et al., 2015, PLoS One 10). In silico searches for potential miR-17-5p and miR-21-5p target sites revealed 5 occult miR-17-3p passenger strand targets in the 3′UTR of human PTEN mRNA and 6 passenger strand targets in the 3′UTR of PDCD4 mRNA (Jin et al., 2015, PLoS One 10) (FIG. 2). Thus, anti-miR-17-5p apparently attacked the miR-17-3p passenger strand targets in PTEN and PDCD4 mRNA. Conforming to conventional wisdom, no miR-21-3p passenger strand targets were found in PTEN or PDCD4 mRNAs, and anti-miR-21-5p LNA did not alter PDCD4 mRNA translation in MDA-MB-231 cells (Jin et al., 2015, PLoS One 10). A few other passenger strands have been found active, as exceptions to the rule (Mah et al., 2010, Crit Rev Eukaryot Gene Expr 20(2):141-8). Molecular dynamics calculation indicated that miR-17-3p passenger strand can hybridize stably to 3′UTR targets, forming a dynamic A-form helix, without external bulges (FIG. 6) (Jin et al., 2015, PLoS One 10).

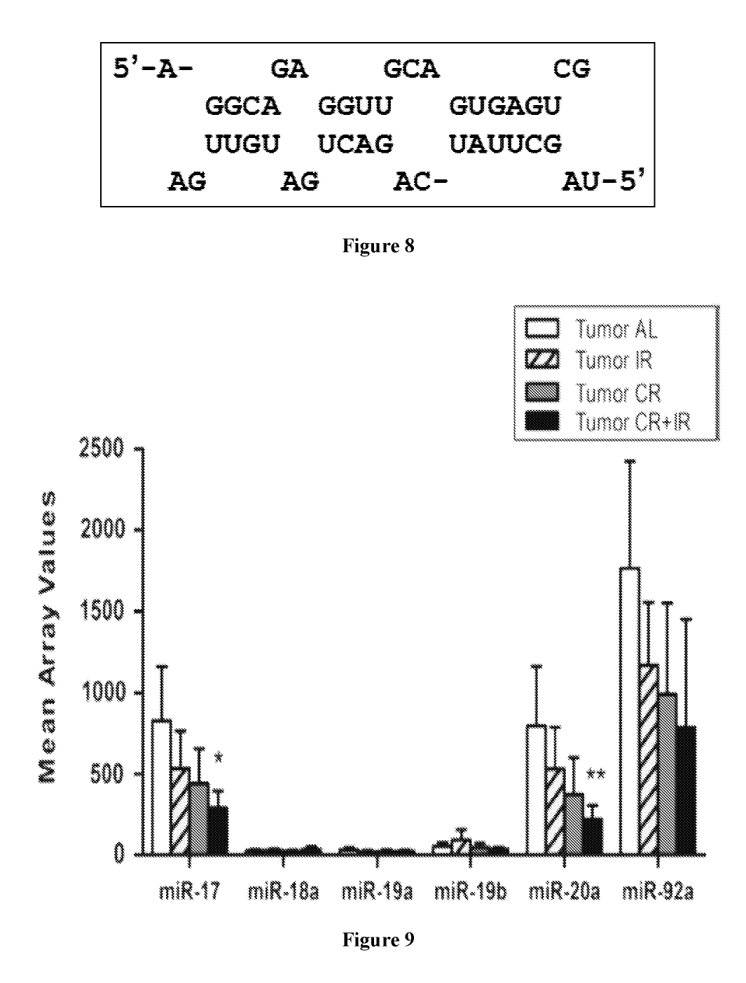

miRNA guide strands hybridized with their passenger strands (FIG. 7), or with 3′UTR targets (FIG. 8), are displayed with bulged mismatches and flipped out extra bases. Those pictures imply terribly weak basepairing, suggesting that miRNA binding should have little regulatory effect. In a crystal structure of human Ago2 with miR-122 and complementary RNA 11mers, Ago2 first props up the miRNA seed region for optimal stacking for RNA hybridization, then locks in the smooth RNA duplex (Schirle et al., 2014, Science 346(6209):608-613). Molecular dynamics calculation of miR-17-3p bound to a 3′UTR site in PTEN mRNA (FIG. 6) predicted stable A-form duplexes for all passenger strand:mRNA targets, as well as for guide strands, despite the mismatches and bulges that appear so distorted in the Mfold presentation. Thus one realizes that miRNA:mRNA duplexes could be accommodated in the substrate groove of Ago2, in agreement with an earlier simulation of an 11mer duplex bound to Thermus thermophilus Ago (Xia et al., 2012, Sci Rep 2:569).

### Example 5

### Caloric Restriction and Ionizing Radiation Down-Regulated miRs in the miR-17˜92 Cluster

Caloric restriction and ionizing radiation down-regulated members of the miR-17˜92 cluster in mouse 4T1 tumors that model triple negative breast cancer (Jin et al., 2014, Breast Cancer Res Treat 146(1):41-50) (FIG. 9). Intervention decreased 4T1 metastatic activities, mainly by suppressing extracellular matrix (ECM) mRNAs that exhibit miR-17-5p binding sites, and c-Myc expression (Jin et al., 2014, Breast Cancer Res Treat 146(1):41-50). This result underscored the importance of miR-17-5p in cell phenotypes.

### Example 6

### DNA-LNA Chimeras Knocked Down Targeted miRs

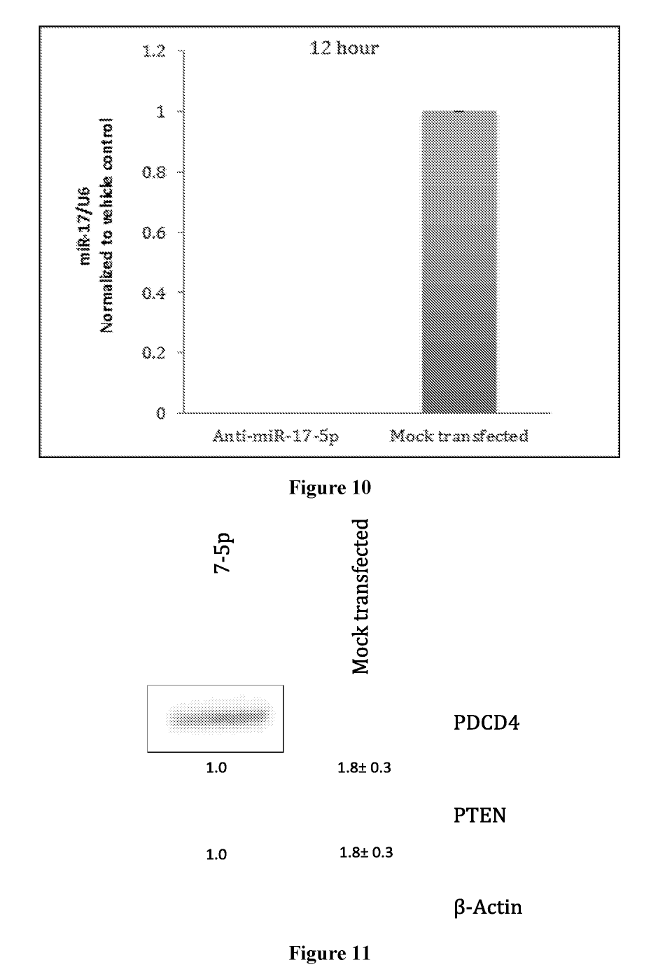

When anti-miR-17-5p DNA-LNA chimera was transfected into human MDA-MB-231 cells, triplicate qPCR revealed 99±0.01% knockdown of miR-17-5p after 12 hr. Similarly, anti-miR-21-5p knocked down miR-17-5p by 99±0.04% after 12 hr (Jin et al., 2015, PLoS One 10) (FIG. 10).

### Example 7

### Anti-miR-17-5p Knockdown of miR-17-5p Unexpectedly Decreased PTEN and PDCD4 Proteins

PTEN mRNA is a known direct target of miR-17-5p (Xiao et al., 2008, Nat Immunol 9(4):405-14). PTEN mRNA was significantly decreased by 15±4% at 12 hr and 22±6% at 48 hr after anti-miR-17-5p transfection (Jin et al., 2015, PLoS One 10). But miR-17-5p knockdown induced no change in PDCD4 mRNA compared to control. When protein levels were analyzed 48 hr after transfection with anti-miR-17-5p, triplicate western blots showed that the PTEN and PDCD4 proteins were down-regulated by 1.8±0.3 fold (FIG. 10), instead of being up-regulated as expected following miR-17-5p knockdown (Jin et al., 2015, PLoS One 10), because miRs typically block translation.

### Example 8

### miR-17-3p Passenger Strand is a Potential Inhibitor of PTEN and PDCD4 mRNAs, as Well as miR-17-5p

Using rna22 (Loher and Rigoutsos, 2012, Bioinformatics 28(24):3322-3), miR-17-5p was identified as a potential PDCD4 mRNA regulator through its interaction with a single site in the 3′UTR (Jin et al., 2015, PLoS One 10). Although rna22 is the only algorithm that predicted a binding site for miR-17-5p in the 3′UTR of PDCD4 mRNA, the predicted 23 bp miRNA:mRNA duplex is stable, containing 17 complementary basepairs and an Mfold predicted folding energy ΔG° of −24.5 kcal/mol at 37° C. To understand the unexpected reduction of PTEN and PDCD4 in FIG. 11, miR-17 was examined in miRBase. miR-17-5p was predicted to exist in a duplex with its passenger strand miR-17-3p in the pre-miRNA hairpin structure (FIG. 7). Most of the miR-17-3p is fully complementary to its guide strand miR-17-5p, especially in the seed sequence (nt 2-8) of miR-17-3p. Since anti-miR-17-5p is fully complementary to miR-17-5p, its sequence is therefore highly homologous to miR-17-3p (FIG. 7). Therefore, it was predicted that anti-miR-17-5p could act as a miR-1′7-3p mimic, binding to miR-17-3p target sites in the 3′UTR of PDCD4 and PTEN mRNAs.

### Example 9

### Silencing the miR-17-3p Passenger Strand Maintained PDCD4 and PTEN Protein Levels

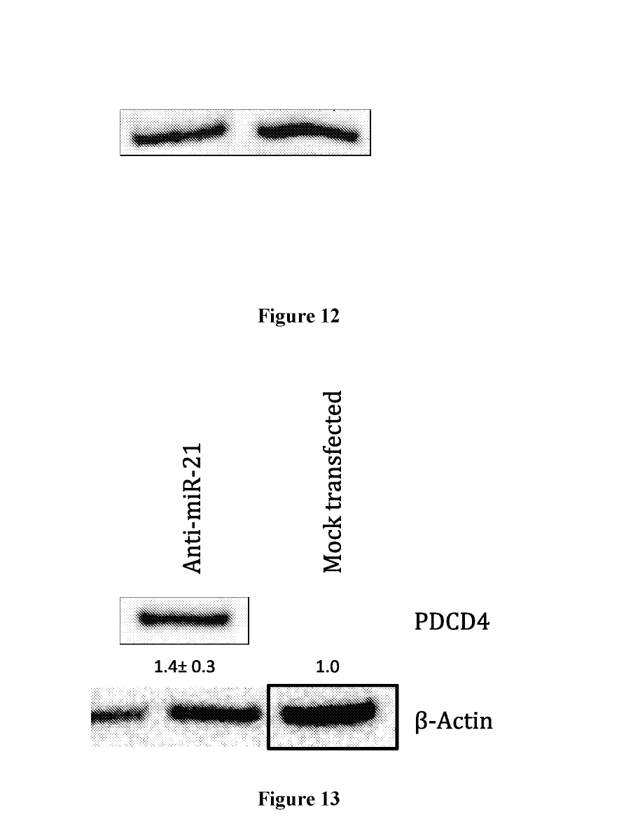

To determine if the passenger strand caused the contradictory results above, we knocked down endogenous miR-17-3p with anti-miR-17-3p, then analyzed PDCD4 and PTEN protein levels. In contrast to miR-17-5p knockdown (FIG. 11), triplicate western blots after miR-17-3p knockdown showed no significant changes in PDCD4 or PTEN protein levels (FIG. 12) (Jin et al., 2015, PLoS One 10). The maintained protein levels of PDCD4 and PTEN could be a comprehensive outcome of both miR-17-5p and miR-17-3p binding to the PDCD4 and PTEN3′UTRs (FIG. 2). The static result is plausible, because there are more potential binding sites for miR-17-3p on the 3′UTR of PDCD4 and PTEN mRNAs compared to one for miR-17-5p, although anti-miR-17-3p could act as a miR-17-5p mimic (FIG. 7).

### Example 10

### miR-21-5p Guide Strand Knockdown Increased PDCD4 mRNA Level and Elevated PDCD4 Protein Level

To further test the hypothesis, anti-miR-21-5p was transfected into MDA-MB-231 cells. rna22, Targetscan (Lewis et al., 2005, Cell 120(1):15-20), and miRanda predicted that miR-21-5p has 2 binding sites in the 3′UTR of PDCD4 mRNA, while its passenger strand miR-21-3p has no putative binding sites, unlike miR-17-3p. Anti-miR-21-5p knocked down miR-21-5p by 96±0.15%, and increased PDCD4 mRNA by 33±9.6% at 12 hr, and 17±3.3% at 48 hr. Importantly, miR-21-5p knockdown increased PDCD4 protein expression by 1.4±0.3 fold (FIG. 13) (Jin et al., 2015, PLoS One 10). Consistent with the absence of a miR-21-3p site on PDCD4 mRNA, anti-miR-21-5p did not down-regulate PDCD4 protein.

Thus, anti-miR-17-5p mimicked miR-17-3p, and anti-miR-17-3p mimicked miR-17-5p. These results indicate that therapeutic silencing sequences should be designed to target the miRNA strand with the greatest number of putative binding sites in the 3′UTRs of target mRNAs, while minimizing affinity for the minor strand.

### Example 11

### Luciferase Vector Construction

Given the prospect of passenger strand targets in the 3′UTRs of PDCD4 and PTEN mRNAs, luciferase vectors have been constructed to report effects on individual sites in the 3′UTRs of PDCD4 and PTEN mRNAs. pMir-Report-Luciferase is the base. Synthetic DNA 60mers were inserted into the vectors for each of the predicted 3′UTR targets:

miR-17-5p PDCD4
17-5pPDCD4 S
(SEQ ID NO: 49)

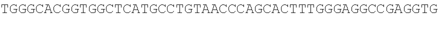

GGGG

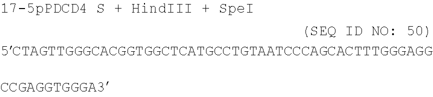

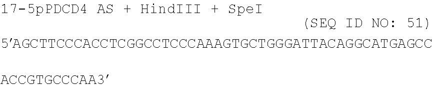

miR-17-5p PTEN
17-5p PTEN S
(SEQ ID NO: 52)

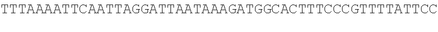

AGT

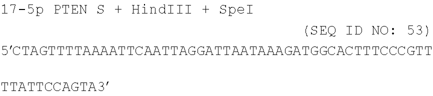

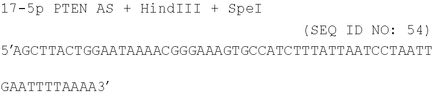

miR-17-3p PDCD4
17-3p PDCD4 51
(SEQ ID NO: 55)
AAACCAGAGAGCTACTGAATATAAGAACTCTTGCAGTCTTAGATGTTATA
AA

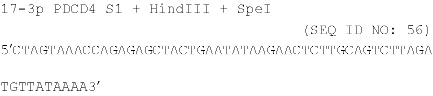

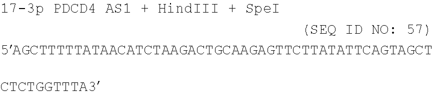

17-3p PDCD4 S2
(SEQ ID NO: 58)
GCCACTCCTTTCTTTCAAGGACAGTGTTTTTTGTAGTAAAATCACTGGTT
TA

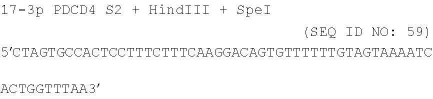

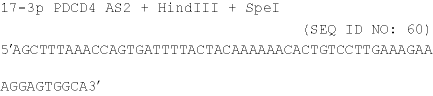

17-3p PDCD4 S3
(SEQ ID NO: 61)
ACGTCTGTGCTAATTTAAACTGCCAAATATTGACTGCAGCAAACAAGAAT
TAT

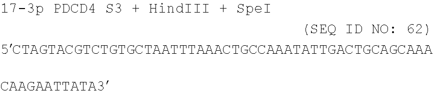

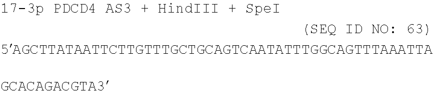

17-3p PDCD4 S4
(SEQ ID NO: 64)
GGAGAATTGCTTGAACCTGGGAGGCAGAGGTTGCAGTGAGTCGAGATGG
TGC

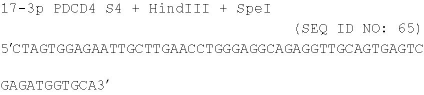

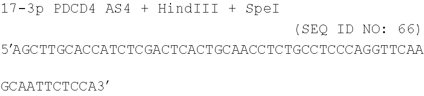

miR-17-3p PTEN
17-3p PTEN 51
(SEQ ID NO: 67)

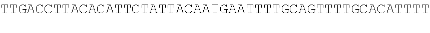

TTA

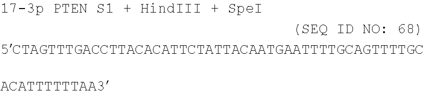

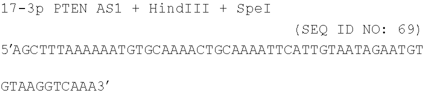

17-3p PTEN S2
(SEQ ID NO: 70)

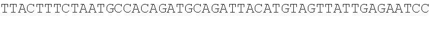

TTT

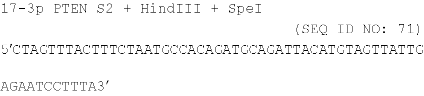

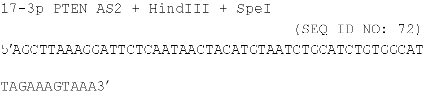

17-3p PTEN S3
(SEQ ID NO: 73)

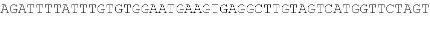

GTT

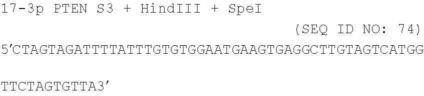

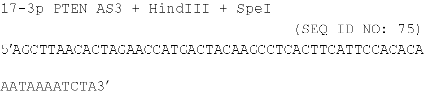

17-3p PTEN S4
(SEQ ID NO: 76)

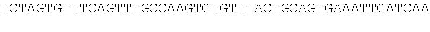

ATG

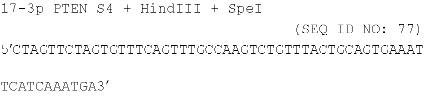

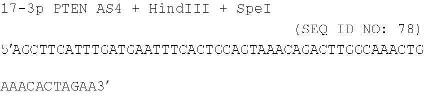

17-3p PTEN S5
(SEQ ID NO: 79)

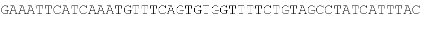

TGG

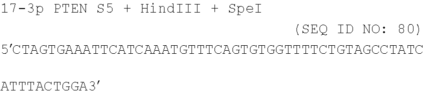

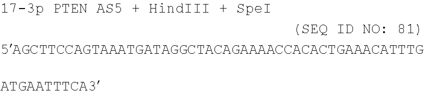

17-3p PTEN S6
(SEQ ID NO: 82)

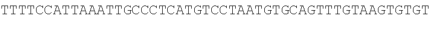

GTG

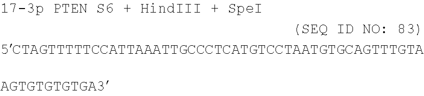

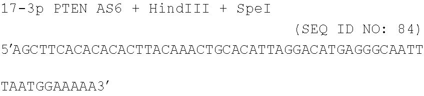

Annealed duplexes were ligated into Hind III-Spe I linearized pMir-Report-Luciferase, then used to transform E. coli DH5α. Individual colonies were grown up overnight in Terrific Broth. Plasmids were isolated, then sequenced across the insert zone.

### Example 12

### Peptide Nucleic Acid (PNA) Hybridization to RNA

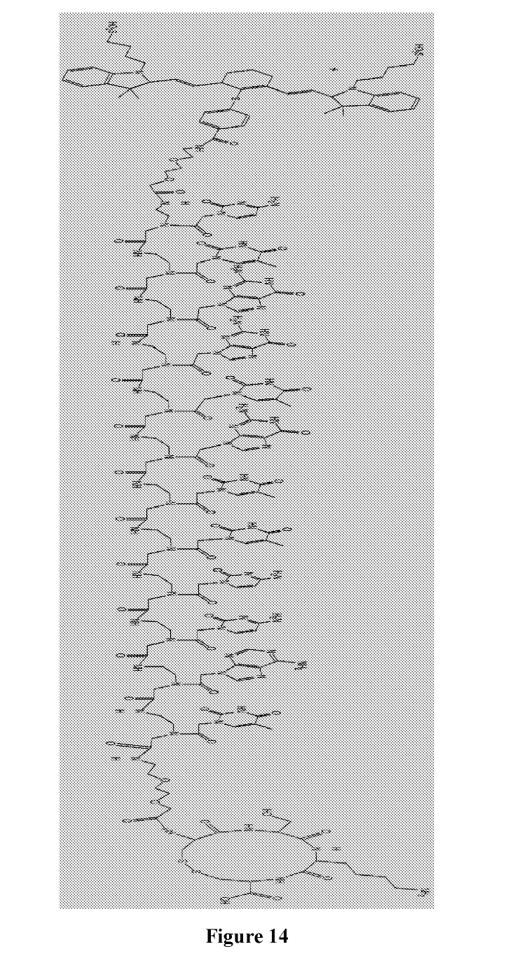

Peptide nucleic acid (PNA), a nuclease-resistant polyamide derivative that binds tightly to RNA targets with single mismatch specificity (Chakrabarti et al., 2007, Cancer Biology & Therapy 6(6):948-956), was also utilized for mRNA binding in cells (FIG. 14).

RISC and RNase H fail to recognize PNA, so that PNA can bind to RNAs in cells, but not ablate them (Good and Nielsen, 1997, Antisense Nucleic Acid Drug Dev 7(4):431-7; Tian et al., 2003, Annals of the New York Academy of Sciences 1002:165-188). Due to their uncharged backbones, PNAs hybridize to RNA more strongly and specifically than most oligonucleotide derivatives (Good and Nielsen, 1997, Antisense Nucleic Acid Drug Dev 7(4):431-7), comparable to LNA. Experience to date with PNA implies that the initiation codon region is the most effective region to probe (Good and Nielsen, 1997, Antisense Nucleic Acid Drug Dev 7(4):431-7).

Unconjugated PNAs are not significantly taken up by cells (Gray et al., 1997, Biochemical Pharmacology 53(10):1465-1476). To enable RNA blocking in cultured cells by antisense PNA, without transfection or electroporation, PNA oligomers were designed with a D(CSKC) tetrapeptide analog of insulin-like growth factor 1 (IGF1) at the C-terminus of PNA to direct endocytosis into cells that overexpress IGF1R (Basu and Wickstrom, 1997, Bioconjugate Chemistry 8(4):481-488).

It was discovered that 12mer PNA-D(CSKC) sequences, theoretically unique among transcribed sequences (Hélène and Toulmé, 1990, Biochim Biophys Acta 1049(2):99-125), displayed melting temperatures of ≈80° C. with complementary RNA at 1 μM strands in physiological salt, sufficient for hybridization with 1-10 nM mRNAs in cells of tumors with single mismatch specificity (Tian and Wickstrom, 2002, Organic Letters 4(23):4013-4016).

### Example 13

### PNA-Peptide Blocking of miR-17-5p in Cells without Transfection

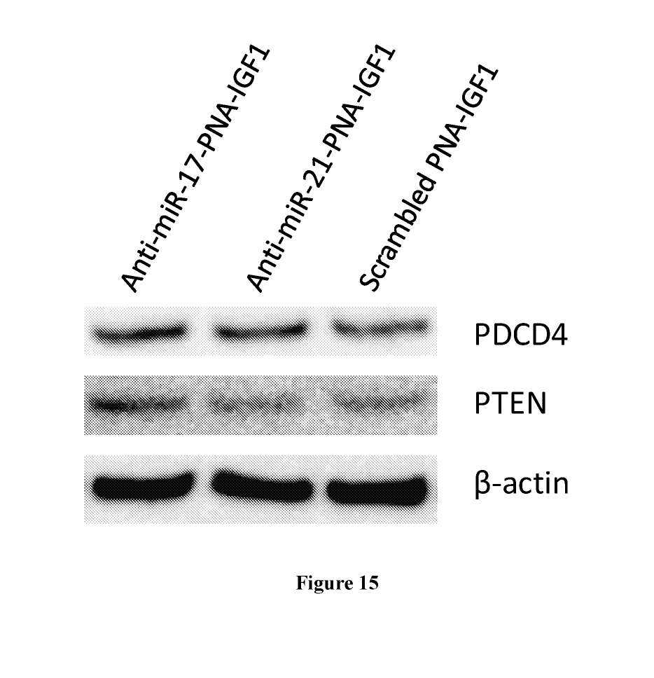

Anti-miR-17-5p PNA-D(CSKC) increased the expression of PDCD4 and PTEN proteins (FIG. 15). Anti-miR-21-5p PNA-D(CSKC) slightly increased the protein expression of PDCD4. Thus, the PNAs acted outside of RISC, blocking miRNA behavior, without the opportunity to imitate the opposing miRNA strand.

### Example 14

### Utilizing the Knowledge of Passenger Strand Activity to Design Unambiguous Knockdown Agents

Based on the deduction of passenger strand activity against particular mRNA 3′UTRs anti-miR sequences, targeting either the guide or passenger strands, will be complementary to the miR seed sequences, and adjacent nucleotides that differ from the opposing strand, with resulting minimal off-target effects, due to the shortness of the interfering PNA. BLAST analyses can be used to reveal the potential for interaction with non-targeted RNAs (Altschul et al., 1997, Nucleic Acids Res 25(17):3389-402). Strand selection rules that acknowledge the potential for passenger strand targets and activity can be used to design functional mimics of passenger strands and guide strands to reverse pathogenic states, free of confounding activities (e.g., passenger strand activity against particular mRNA 3′UTRs). Therapeutic silencing sequences should be designed to target the miRNA strand with the greatest number of putative binding sites in the 3′UTRs of target mRNAs, while minimizing affinity for the minor strand.

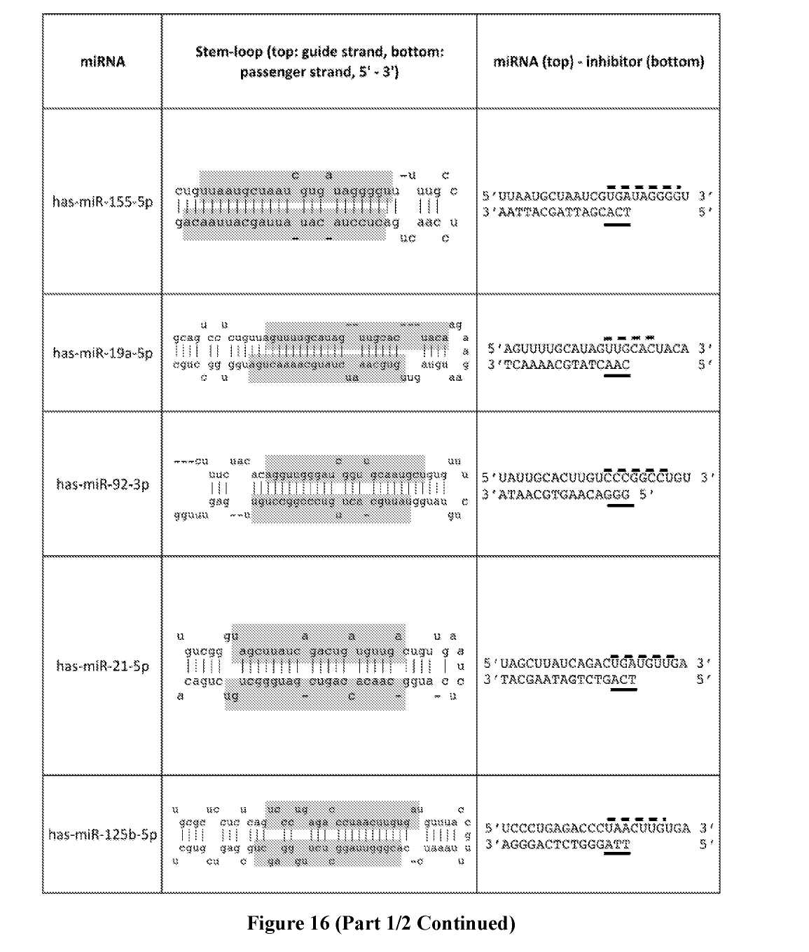

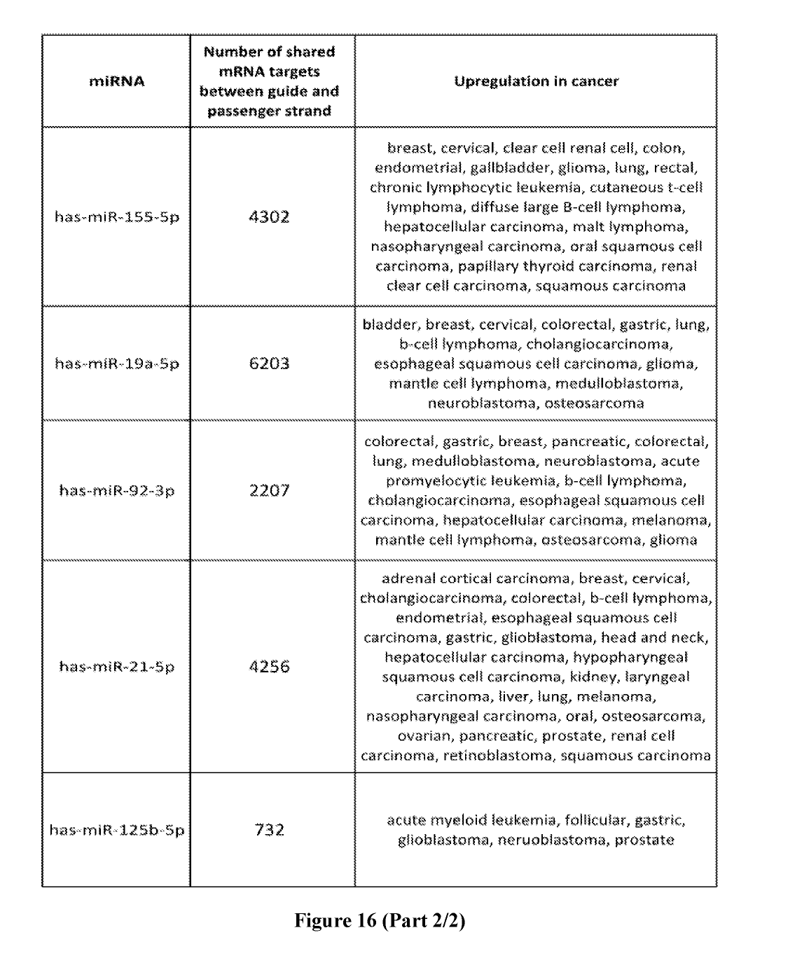

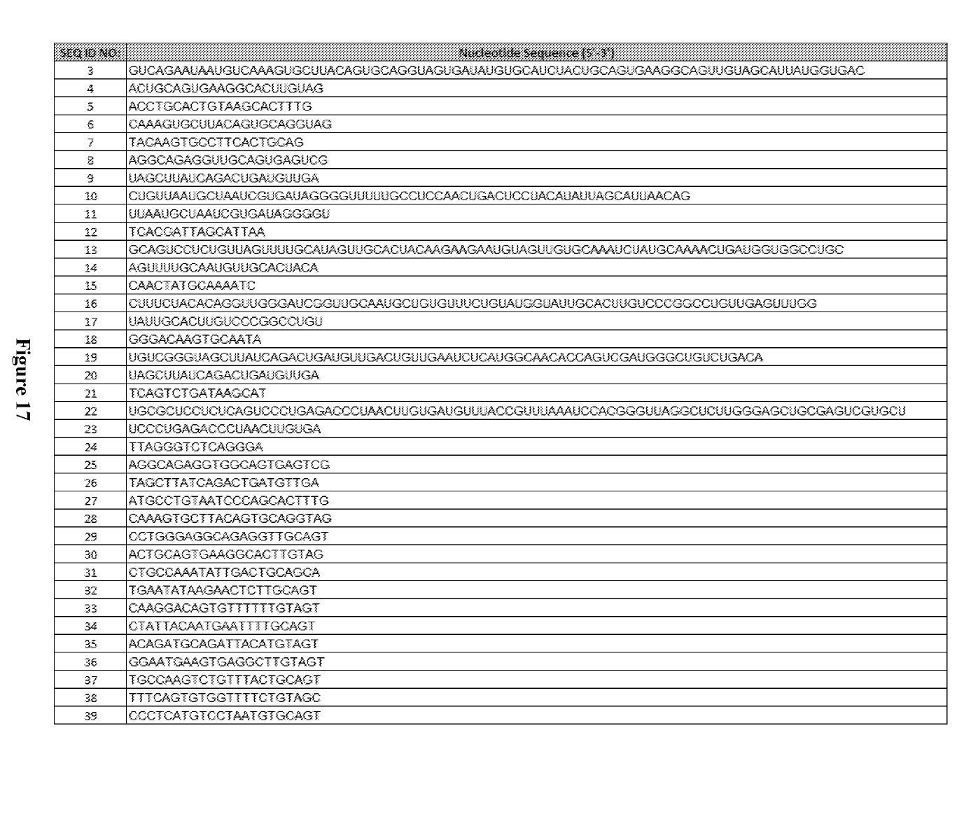

For example, miRNA inhibitors according to the invention were designed for various miRNAs (FIG. 16). As depicted, the stem-loop structure for each miRNA shows the complementarity between the guide strand (top sequence highlighted in grey) and the passenger strand (bottom sequence highlighted in grey). Each miRNA, which the inhibitor is designed for, is shown as the top sequence, whereas the sequence marked with dashed lines represents nucleotides that are complementary to the seed region of the other strand. The inhibitor sequence is shown as the bottom sequence, whereas underlined part of the sequence represents possible extension of the inhibitor sequence to include several nucleotides into the seed sequence of the other strand. The shared targets between the guide and the passenger strands are predicted by miRWalk, DIANA-mT, miRanda, miRDB, PICTAR, PITA, rna22, and TargetScan with minimum of 6 seed pairing. Cancer association for selected miRNAs is summarized from miRCancer database.

Analysis of miRNA inhibitors using Mfold prediction shows that guide and passenger strand inhibitors have the potential to mimic passenger and guide strands, respectively. Mfold prediction shows that anti-miR-17-5p DNA-LNA can mimic miR-17-3p and binds to miR-17-3p target sites in the 3′UTR of PDCD4 (Table 2) and PTEN mRNAs (Table 3). Mfold prediction also shows that anti-miR-17-3p DNA-LNA can mimic miR-17-5p and binds to miR-17-5p target sites in the 3′UTR of PDCD4 and PTEN mRNAs. For Tables 2-4, the DNA-LNA inhibitor sequence is shown as the bottom strand of each duplex, and the top strand of each duplex is the predicted target sequence in the 3′UTR of mRNAs.

TABLE 2
Mfold prediction of anti-miR-17-5p binding to miR-17-3p
target sites in the PDCD4-ENST00000280154

| Position | Predicted Target Sequence | Folding Energy AG | Algorithm |
|---|---|---|---|
| 13##19 | UGAAUAUAAGAACU\| | −7.6 | rna22 |
| | CUUGCAGU | | |
| | GAAUGUCA | | |
| | GUUUCAAC-------\hat{ } CGUCCA | | |
| 325##246 | CAAGGAC UU--\| U | −8.5 | rna22 |
| | AGUGUUU UGUAG | | |
| | UCACGAA ACGUC | | |
| | GUU---- UGUC\hat{ } CA | | |
| 1110##136 | UU\| C CAAA UGAC C | −4.9 | Miranda# |
| | UAAA UGC UAU UGCAG | | rna22 |
| | GUUU ACG AUG ACGUC | | |
| | --\hat{ } C A--- UC-- CA | | |
| 1631##652 | CCUG - AGA-\| U U | −9.5 | rna22 |
| | GGAG GC GGU GCAG | | |
| | UUUC CG UCA CGUC | | |
| | G--- A AAUG\hat{ } - CA | | |

(SEQ ID NO: 85)
5′-UGAAUAUAAGAACUCUUGCAGU-3′
(SEQ ID NO: 86)
5′-CAAGGACAGUGUUUUUUGUAGU-3′
(SEQ ID NO: 87)
5′-UUUAAACUGCCAAAUAUUGACUGCAGC-3′
(SEQ ID NO: 88)
5′-CCUGGGAGGCAGAGGUUGCAGU-3′
(SEQ ID NO: 89, miR-17-3p)
5′-ACCUGCACUGUAAGCACUUUG-3′

TABLE 3
Mfold prediction of anti-miR-17-5p binding to miR-17-3p
target sites found in the 3′UTR of PTEN mRNA.

| Position | Predicted Target Sequence | Folding Energy AG | Algorithm |
|---|---|---|---|
| 1199##220 | CUAUUACAAUGAAU\| | −5.8 | rna22 |
| | UUUGCAGU | | |
| | GAAUGUCA | | |
| | GUUUCAC-------\hat{ } CGUCCA | | |
| 5075##5096 | A -\| AGA - U | −9.3 | rna22 |
| | CAGA UGC UUACA UGUAG | | |
| | GUUU ACG AAUGU ACGUC | | |
| | - C\hat{ } --- C CA | | |
| 5828##5849 | GGAAU AG- U\| U | !10.5 | rna22 |
| | GAAGUG GC UGUAG | | |
| | UUUCAC UG ACGUC | | |
| | G---- GAA UC\hat{ } CA | | |
| 5871##5892 | UGC UC --\| U | !10.7 | rna22 |
| | CAAG UGUUUAC UGCAG | | |
| | GUUU ACGAAUG ACGUC | | |
| | --- C- UC\hat{ } CA | | |
| 5908##5928 | UUUC\| GGUUUUC C | !6.8 | rna22 |
| | AGUGU UGUAG | | |
| | UCACG ACGUC | | |
| | GUU-\hat{ } AAUGUC- CA | | |
| 6059##6080 | CCCUCA\| CCUAAU U | !7.9 | rna22 |
| | UGU GUGCAG | | |
| | ACG CACGUC | | |
| | GUUUC-\hat{ } AAUGU- CA | | |

(SEQ ID NO: 90)
5′-CUAUUACAAUGAAUUUUGCAGU-3′
(SEQ ID NO: 91)
5′-ACAGAUGCAGAUUACAUGUAGU-3′
(SEQ ID NO: 92)
5′-GGAAUGAAGUGAGGCUUGUAGU-3′
(SEQ ID NO: 93)
5′-UGCCAAGUCUGUUUACUGCAGU-3′
(SEQ ID NO: 94)
5′-UUUCAGUGUGGUUUUCUGUAGC-3′
(SEQ ID NO: 95)
5′-CCCUCAUGUCCUAAUGUGCAGU-3′
(SEQ ID NO: 89, miR-17-3p)
5′-ACCUGCACUGUAAGCACUUUG-3′

TABLE 4
Mfold prediction of anti-miR-17-3p binding to miR-17-5p
target sites in the 3′UTR of PDCD4 and PTEN mRNAs.

| | | | |
|---|---|---|---|
| Position | Predicted Target Sequence | Folding Energy AG | Algorithm |

PDCD4-ENST00000280154

| | | | |
|---|---|---|---|
| 1446$$1468 | AUGC\| AUCCCA U | −9.3 | rna22 |
| | CUGUA GCACUU G | | |
| | GACGU CGUGAA C | | |
| | ----\hat{ } CACUUC - AU | | |

PTEN-ENST00000371953

| | | | |
|---|---|---|---|
| 257$$279 | GGAUUAAUAAA\| U UC | −10.4 | Targetscan |
| | GA GGCACUU | | Miranda |
| | CU CCGUGAA | | |
| | GACGUCA----\hat{ } U CAU | | |

(SEQ ID NO: 96)
5′-AUGCCUGUAAUCCCAGCACUUUG-3′
(SEQ ID NO: 97)
5′-GGAUUAAUAAAGAUGGCACUUUC-3′
(SEQ ID NO: 98, miR-17-5p)
5′-UACAAGUGCCUUCACUGCAG-3′

Preliminary results support the hypotheses for LNA knockdown of miRNAs in transformed cells. These results also imply activity by receptor-mediated endocytosis of PNA-D(CSKC). It is postulated that DNA-LNA-peptides also accumulate preferentially in cells that overexpress a receptor that binds a selected peptide ligand.

Plasma binding proteins that carry IGF1 (Ellis et al., 1998, Breast Cancer Res Treat 52(1-3):175-84) provide favorable systemic pharmacokinetics for reporter-PNA-D(CSKC) (Opitz et al., 2010, Oligonucleotides 20(3):117-125), even though PNAs by themselves are eliminated quickly due to poor plasma protein binding (Gray and Wickstrom, 1997, Antisense and Nucleic Acid Drug Development 7(3):133-140). PNA-peptides, at 2.5 mg/kg in mice, displayed no toxicity (Boffa et al., 2005, Oligonucleotides 15(2):85-93), immunogenicity (Cutrona et al., 2007, Oligonucleotides 17(1):146-50), mutagenicity, or elastogenicity (Boffa et al., 2007, Cancer Gene Ther 14(2):220-6).

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated inhibitory nucleic acid that is fully complementary to at least 50% of a microRNA (miRNA) strand, but no more than 75% of the miRNA strand, starting at the 5′ region of the miRNA strand.

2. The isolated inhibitory nucleic acid of claim 1, wherein the miRNA strand is a guide strand or passenger strand.

3. The isolated inhibitory nucleic acid of claim 1, wherein the inhibitory nucleic acid is DNA or RNA.

4. The isolated inhibitory nucleic acid of claim 1, comprising one or more modifications selected from phosphorothioate, morpholino phosphoramidate, methylphosphonate, boranophosphate, locked nucleic acid, peptide nucleic acid, 2′-fluoro, 2′-amino, 2′-thio, or 2′-O-alkyl.

5. The isolated inhibitory nucleic acid of claim 1, wherein the inhibitory nucleic acid specifically binds the seed region of the miRNA guide strand.

6. The isolated inhibitory nucleic acid of claim 1, wherein the inhibitory nucleic acid excludes the sequence of the seed region of the miRNA passenger strand.

7. The isolated inhibitory nucleic acid of claim 1, wherein the miRNA is miR-17 or miR-21.

8. The isolated inhibitory nucleic acid of claim 7, wherein the inhibitory nucleic acid comprises the nucleic acid sequence 5′-GTAAGCACTTTG-3′(SEQ ID NO: 1) and binds miR-17-5p.

9. The isolated inhibitory nucleic acid of claim 7, wherein the inhibitory nucleic acid comprises the nucleic acid sequence 5′-TCTGATAAGCTA-3′(SEQ ID NO: 2) and binds miR-21-5p.

10. The isolated inhibitory nucleic acid of claim 7, wherein the inhibitory nucleic acid does not bind to a PTEN or PDCD4 mRNA.

11. A method for treating neoplasia in a subject, the method comprising administering to the subject an effective amount of the inhibitory nucleic acid of claim 1 that binds to miR-17-5p or miR-21-5p.

12. The method of claim 11, wherein the inhibitory nucleic acid does not bind to a PTEN or PDCD4 mRNA.

13. The method of claim 11, wherein the inhibitory nucleic acid is DNA or RNA.

14. The method of claim 11, wherein the inhibitory nucleic acid comprises one or more modifications selected from phosphorothioate, morpholino phosphoramidate, methylphosphonate, boranophosphate, locked nucleic acid, peptide nucleic acid, 2′-fluoro, 2′-amino, 2′-thio, or 2′-O-alkyl.

15. The method of claim 11, wherein the inhibitory nucleic acid specifically binds the seed region of the targeted miRNA strand.

16. The method of claim 15, wherein the inhibitory nucleic acid includes up to three bases of the seed region of the opposite miRNA strand.

17. The method of claim 11, wherein the inhibitory nucleic acid comprises the nucleic acid sequence 5′-GTAAGCACTTTG-3′ (SEQ ID NO: 1) and binds miR-17-5p.

18. The method of claim 11, wherein the inhibitory nucleic acid comprises the nucleic acid sequence 5′-TCTGATAAGCTA-3′(SEQ ID NO: 2) and binds miR-21-5p.

19. The method of claim 11, wherein the neoplasm is breast cancer, including triple negative breast cancer.

20. A method of decreasing binding of an miRNA to an mRNA in a cell, the method comprising administering to the cell an inhibitory nucleic acid that is fully complementary to at least 50% of a microRNA (miRNA) strand, but no more than 75% of the miRNA strand, starting at the 5′ region of the miRNA strand.

21. The method of claim 20, wherein the miRNA strand is a guide strand or passenger strand.

22. The method of claim 20, wherein the inhibitory nucleic acid does not bind or minimizes binding to the mRNA.

23. The method of claim 20, wherein the mRNA is a PTEN or PDCD4 mRNA.

24. The method of claim 23, wherein the inhibitory nucleic acid binds to miR-17-5p or miR-21-5p.

25. The method of claim 20, wherein the inhibitory nucleic acid is DNA or RNA.

26. The method of claim 20, wherein the inhibitory nucleic acid comprises one or more modifications selected from phosphorothioate, morpholino phosphoramidate, methylphosphonate, boranophosphate, locked nucleic acid, peptide nucleic acid, 2′-fluoro, 2′-amino, 2′-thio, or 2′-O-alkyl.

27. The method of claim 20, wherein the inhibitory nucleic acid specifically binds the seed region of the targeted miRNA strand.

28. The method of claim 27, wherein the inhibitory nucleic acid excludes the sequence of the seed region of the opposite miRNA strand.

29. The method of claim 20, wherein the inhibitory nucleic acid comprises the nucleic acid sequence 5′-GTAAGCACTTTG-3′(SEQ ID NO: 1) and binds miR-17-5p.

30. The method of claim 20, wherein the inhibitory nucleic acid comprises the nucleic acid sequence 5′-TCTGATAAGCTA-3′(SEQ ID NO: 2) and binds miR-21-5p.

31. The method of claim 20, wherein the cell is a breast cancer or triple negative breast cancer cell.


SEQUENCE LISTING (99 sequences)

SEQ ID NO: 1 - DNA, 12 bp, Artificial Sequence
  Artificially Synthesized
gtaagcactttg

SEQ ID NO: 2 - DNA, 12 bp, Artificial Sequence
  Artificially Synthesized
tctgataagcta

SEQ ID NO: 3 - RNA, 84 nt, Homo sapiens
gucagaauaaugucaaagugcuuacagugcagguagugauaugugcaucuacugcagugaaggcaguuguagcauuauggugac

SEQ ID NO: 4 - RNA, 22 nt, Homo sapiens
acugcagugaaggcacuuguag

SEQ ID NO: 5 - DNA, 21 bp, Artificial Sequence
  Artificially Synthesized
acctgcactgtaagcactttg

SEQ ID NO: 6 - RNA, 23 nt, Homo sapiens
caaagugcuuacagugcagguag

SEQ ID NO: 7 - DNA, 20 bp, Artificial Sequence
  Artificially Synthesized
tacaagtgccttcactgcag

SEQ ID NO: 8 - RNA, 22 nt, Homo sapiens
aggcagagguugcagugagucg

SEQ ID NO: 9 - RNA, 22 nt, Homo sapiens
uagcuuaucagacugauguuga

SEQ ID NO: 10 - RNA, 65 nt, Homo sapiens
cuguuaaugcuaaucgugauagggguuuuugccuccaacugacuccuacauauuagcauuaacag

SEQ ID NO: 11 - RNA, 23 nt, Homo sapiens
uuaaugcuaaucgugauaggggu

SEQ ID NO: 12 - DNA, 16 bp, Artificial Sequence
  Artificially Synthesized
tcacgattagcattaa

SEQ ID NO: 13 - RNA, 82 nt, Homo sapiens
gcaguccucuguuaguuuugcauaguugcacuacaagaagaauguaguugugcaaaucuaugcaaaacugaugguggccugc

SEQ ID NO: 14 - RNA, 22 nt, Homo sapiens
aguuuugcaauguugcacuaca

SEQ ID NO: 15 - DNA, 15 bp, Artificial Sequence
  Artificially Synthesized
caactatgcaaaatc

SEQ ID NO: 16 - RNA, 78 nt, Homo sapiens
cuuucuacacagguugggaucgguugcaaugcuguguuucuguaugguauugcacuugucccggccuguugaguuugg

SEQ ID NO: 17 - RNA, 22 nt, Homo sapiens
uauugcacuugucccggccugu

SEQ ID NO: 18 - DNA, 15 bp, Homo sapiens
gggacaagtgcaata

SEQ ID NO: 19 - RNA, 72 nt, Homo sapiens
ugucggguagcuuaucagacugauguugacuguugaaucucauggcaacaccagucgaugggcugucugaca

SEQ ID NO: 20 - RNA, 22 nt, Homo sapiens
uagcuuaucagacugauguuga

SEQ ID NO: 21 - DNA, 16 bp, Homo sapiens
tcagtctgataagcat

SEQ ID NO: 22 - RNA, 88 nt, Homo sapiens
ugcgcuccucucagucccugagacccuaacuugugauguuuaccguuuaaauccacggguuaggcucuugggagcugcgagucgugcu

SEQ ID NO: 23 - RNA, 22 nt, Homo sapiens
ucccugagacccuaacuuguga

SEQ ID NO: 24 - DNA, 15 bp, Artificial Sequence
  Artificially Synthesized
ttagggtctcaggga

SEQ ID NO: 25 - DNA, 22 bp, Homo sapiens
aggcagaggtggcagtgagtcg

SEQ ID NO: 26 - DNA, 22 bp, Homo sapiens
tagcttatcagactgatgttga

SEQ ID NO: 27 - DNA, 23 bp, Homo sapiens
atgcctgtaatcccagcactttg

SEQ ID NO: 28 - DNA, 23 bp, Homo sapiens
caaagtgcttacagtgcaggtag

SEQ ID NO: 29 - DNA, 22 bp, Homo sapiens
cctgggaggcagaggttgcagt

SEQ ID NO: 30 - DNA, 22 bp, Homo sapiens
actgcagtgaaggcacttgtag

SEQ ID NO: 31 - DNA, 22 bp, Homo sapiens
ctgccaaatattgactgcagca

SEQ ID NO: 32 - DNA, 22 bp, Homo sapiens
tgaatataagaactcttgcagt

SEQ ID NO: 33 - DNA, 22 bp, Homo sapiens
caaggacagtgttttttgtagt

SEQ ID NO: 34 - DNA, 22 bp, Homo sapiens
ctattacaatgaattttgcagt

SEQ ID NO: 35 - DNA, 22 bp, Homo sapiens
acagatgcagattacatgtagt

SEQ ID NO: 36 - DNA, 22 bp, Homo sapiens
ggaatgaagtgaggcttgtagt

SEQ ID NO: 37 - DNA, 22 bp, Homo sapiens
tgccaagtctgtttactgcagt

SEQ ID NO: 38 - DNA, 22 bp, Homo sapiens
tttcagtgtggttttctgtagc

SEQ ID NO: 39 - DNA, 22 bp, Homo sapiens
ccctcatgtcctaatgtgcagt

SEQ ID NO: 40 - DNA, 84 bp, Homo sapiens
gtcagaataatgtcaaagtgcttacagtgcaggtagtgatatgtgcatctactgcagtgaaggcacttgtagcattatggtgac

SEQ ID NO: 41 - DNA, 72 bp, Homo sapiens
tgtcgggtagcttatcagactgatgttgactgttgaatctcatggcaacaccagtcgatgggctgtctgaca

SEQ ID NO: 42 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 43 - protein, 403 aa, Homo sapiens [sequence omitted]

SEQ ID NO: 44 - protein, 455 aa, Homo sapiens
MDVENEQILNVNPAENAGTEEIKNEINGNWISASSINEARINAKAKRRLRKNSSRDSGRGDSVSDSGSDALRSGLTVPTSPKGRLLDRRSRSGKGRGLPKKGGAGGKGVWGTPGQVYDVEEVDVKDPNYDDDQENCVYETVVLPLDERAFEKTLTPIIQEYFEHGDTNEVAEMLRDLNLGEMKSGVPVLAVSLALEGKASHREMTSKLLSDLCGTVMSTTDVEKSFDKLLKDLPELALDTPRAPQLVGQFIARAVGDGILCNTYIDSYKGTVDCVQARAALDKATVLLSMSKGGKRKDSVWGSGGGQQSVNHLVKEIDMLLKEYLLSGDISEAEHCLKELEVPHFHHELVYEAIIMVLESTGESTFKMILDLLKSLWKSSTITVDQMKRGYERIYNEIPDINLDVPHSYSVLERFVEECFQAGIISKQLRDLCPSRGRKRFVSEGDGGRLKPESY

SEQ ID NO: 45 - DNA, 3602 bp, Homo sapiens
cttttcctcctcagctccggctccgccgccacgattggccagccgaccacccggcctcggccaataagcgccgccctctcgcccccgtgttactgggtagaagaaaacaaaaacaaacagagcgagaagggccagagactctccgaggcggcggcagagacagaagagcggggtcggggccggctgaccaggaacctgggcgagcagcggcgggggcccgagggattctgaaggaagatttccattaggtaatttgtttaatcagtgcaagcgaaattaagggaaaatggatgtagaaaatgagcagatactgaatgtaaaccctgcagaaaatgctgggactgaggaaataaagaatgaaataaatggaaattggatttcagcatcctccattaacgaagctagaattaatgccaaggcaaaaaggcgactaaggaaaaactcatcccgggactctggcagaggcgattcggtcagcgacagtgggagtgacgcccttagaagtggattaactgtgccaaccagtccaaagggaaggttgctggataggcgatccagatctgggaaaggaaggggactaccaaagaaaggtggtgcaggaggcaaaggtgtctggggtacacctggacaggtgtatgatgtggaggaggtggatgtgaaagatcctaactatgatgatgaccaggagaactgtgtttatgaaactgtagttttgcctttggatgaaagggcatttgagaagactttaacaccaatcatacaggaatattttgagcatggagatactaatgaagttgcggaaatgttaagagatttaaatcttggtgaaatgaaaagtggagtaccagtgttggcagtatccttagcattggaggggaaggctagtcatagagagatgacatctaagcttctttctgacctttgtgggacagtaatgagcacaactgatgtggaaaaatcatttgataaattgttgaaagatctacctgaattagcactggatactcctagagcaccacagttggtgggccagtttattgctagagctgttggagatggaattttatgtaatacctatattgatagttacaaaggaactgtagattgtgtgcaggctagagctgctctggataaggctaccgtgcttctgagtatgtctaaaggtggaaagcgtaaagatagtgtgtggggctctggaggtgggcagcaatctgtcaatcaccttgttaaagagattgatatgctgctgaaagaatatttactctctggagacatatctgaagctgaacattgccttaaggaactggaagtacctcattttcaccatgagcttgtatatgaagctattataatggttttagagtcaactggagaaagtacatttaagatgattttggatttattaaagtccctttggaagtcttctaccattactgtagaccaaatgaaaagaggttatgagagaatttacaatgaaattccggacattaatctggatgtcccacattcatactctgtgctggagcggtttgtagaagaatgttttcaggctggaataatttccaaacaactcagagatctttgtccttcaaggggcagaaagcgttttgtaagcgaaggagatggaggtcgtcttaaaccagagagctactgaatataagaactcttgcagtcttagatgttataaaaatatatatctgaattgtaagagttgttagcacaagttttttttttttttttttttaagcacttgttttgggtacaaggcatttctgacattttataaacctacatttaaggggaatttttaaaggaaatgttttttcttttttttttgtttttcgagggggcaaggagggacagaaaagtaacctcttcttaagtggaatattctaataagctaccttttgtaagtgccatgtttattatctaatcattccaagttttgcattgatgtctgactgccactcctttctttcaaggacagtgttttttgtagtaaaatcactggtttatacaaagctttatttagggggtaaagttaagctgctaaaaccccatgttggctgctgctgttgagatactgtgctttgggagtaaaaaaagaaagttatttctttgtcttaaagaatttttaaaaaattagtcatgagacttattcatctttccagggaacatactgattggtcttaaaagactagacagttaagtaaaaggtggctggaacatctatttttctacaaaactggaaaaatgaacctggttctagaagaatgtacaccaaaataaaacatgtgaagcagtattgattctttattgggagtacatttttttaggtctcttaaactttaatttcacacagtaaattttgaatctcataaggaagcatatttgaacctagtcaatttaatcttagtgttcccttgaaaactttttttccctacaaaattttaagtgaaaaatacaatagtaaattaagattacactggggaaaaaaatgcaggtatcactttactccattgttatctgacctagagcttaattaagttttagaaatatgtaataccttccatcattccatcatccttaaattctgttaccaaataatggctaatgttacaaaaagttatactccagagacccaaagcttgacatttacctaatgtatgagaaaatattaccaattaacaataaagaatgatcatatttttaacctcttttacatagcctaataactcagcaaggcctcaacgtctgtgctaatttaaactgccaaatattgactgcagcaaacaagaattatattcagaatttatgagggtactgttaggagtatactgcttacaggtttagatatagtctgttagaattaaaaccaagtttagtgttcatatttacctcatgggctttatcaagcccatattacctcagcttatatatagttaccatttttaggtttttaattgtttgacacttggatgataaatgcagtcattttattctcaagtgcttaaaattaatgtaattaaaagcttagctgactacagaataggtgagggtttcttaaaaatgagatttaagggctgggcacggtggctcatgcctgtaatcccagcactttgggaggccgaggtgggcggatcacttgaggttgggagttcatgaccagcttgaccaacatgaagaaaccctgtctctattaaaaatacaaaagtagccaggcatggtggcgcatgcgtgtaatcccagctacttgggaggctgaggcaggagaattgcttgaacctgggaggcagaggttgcagtgagtcgagatggtgccattgctctcgtttgggcaacaagagtgaaactcttgtctcaaaaaaaaaaaaaaatgaggtttaagacagttttgtcattactggtgggatctggtcacacaagatagcattaaacgtgacatggcacataaaattggttaaaaaattttgttttttaattacgtaatgtaaaagcccaacaaacactttatgcaagattggaatgtatcttcaaattcagatttaataaacatgtaaagatcctctgtaaaaaaaaaaaaaaaaaaaaaaaaaaaaaaa

SEQ ID NO: 46 - DNA, 13 bp, Artificial Sequence
  Artificially Synthesized
ntctgataagcta

SEQ ID NO: 47 - DNA, 13 bp, Artificial Sequence
  Artificially Synthesized
ngtaagcactttg

SEQ ID NO: 48 - DNA, 13 bp, Artificial Sequence
  Artificially Synthesized
ntcagatatgcta

SEQ ID NO: 49 - DNA, 53 bp, Artificial Sequence
  Artificially Synthesized
tgggcacggtggctcatgcctgtaatcccagcactttgggaggccgaggtggg

SEQ ID NO: 50 - DNA, 59 bp, Artificial Sequence
  Artificially Synthesized
ctagttgggcacggtggctcatgcctgtaatcccagcactttgggaggccgaggtggga

SEQ ID NO: 51 - DNA, 59 bp, Artificial Sequence
  Artificially Synthesized
agcttcccacctcggcctcccaaagtgctgggattacaggcatgagccaccgtgcccaa

SEQ ID NO: 52 - DNA, 53 bp, Artificial Sequence
  Artificially Synthesized
tttaaaattcaattaggattaataaagatggcactttcccgttttattccagt

SEQ ID NO: 53 - DNA, 59 bp, Artificial Sequence
  Artificially Synthesized
ctagttttaaaattcaattaggattaataaagatggcactttcccgttttattccagta

SEQ ID NO: 54 - DNA, 59 bp, Artificial Sequence
  Artificially Synthesized
agcttactggaataaaacgggaaagtgccatctttattaatcctaattgaattttaaaa

SEQ ID NO: 55 - DNA, 52 bp, Artificial Sequence
  Artificially Synthesized
aaaccagagagctactgaatataagaactcttgcagtcttagatgttataaa

SEQ ID NO: 56 - DNA, 58 bp, Artificial Sequence
  Artificially Synthesized
ctagtaaaccagagagctactgaatataagaactcttgcagtcttagatgttataaaa

SEQ ID NO: 57 - DNA, 58 bp, Artificial Sequence
  Artificially Synthesized
agctttttataacatctaagactgcaagagttcttatattcagtagctctctggttta

SEQ ID NO: 58 - DNA, 52 bp, Artificial Sequence
  Artificially Synthesized
gccactcctttctttcaaggacagtgttttttgtagtaaaatcactggttta

SEQ ID NO: 59 - DNA, 58 bp, Artificial Sequence
  Artificially Synthesized
ctagtgccactcctttctttcaaggacagtgttttttgtagtaaaatcactggtttaa

SEQ ID NO: 60 - DNA, 58 bp, Artificial Sequence
  Artificially Synthesized
agctttaaaccagtgattttactacaaaaaacactgtccttgaaagaaaggagtggca

SEQ ID NO: 61 - DNA, 53 bp, Artificial Sequence
  Artificially Synthesized
acgtctgtgctaatttaaactgccaaatattgactgcagcaaacaagaattat

SEQ ID NO: 62 - DNA, 59 bp, Artificial Sequence
  Artificially Synthesized
ctagtacgtctgtgctaatttaaactgccaaatattgactgcagcaaacaagaattata

SEQ ID NO: 63 - DNA, 59 bp, Artificial Sequence
  Artificially Synthesized
agcttataattcttgtttgctgcagtcaatatttggcagtttaaattagcacagacgta

SEQ ID NO: 64 - DNA, 52 bp, Artificial Sequence
  Artificially Synthesized
ggagaattgcttgaacctgggaggcagaggttgcagtgagtcgagatggtgc

SEQ ID NO: 65 - DNA, 58 bp, Artificial Sequence
  Artificially Synthesized
ctagtggagaattgcttgaacctgggaggcagaggttgcagtgagtcgagatggtgca

SEQ ID NO: 66 - DNA, 57 bp, Artificial Sequence
  Artificially Synthesized
gcttgcaccatctcgactcactgcaacctctgcctcccaggttcaagcaattctcca

SEQ ID NO: 67 - DNA, 52 bp, Artificial Sequence
  Artificially Synthesized
ttgaccttacacattctattacaatgaattttgcagttttgcacatttttta

SEQ ID NO: 68 - DNA, 58 bp, Artificial Sequence
  Artificially Synthesized
ctagtttgaccttacacattctattacaatgaattttgcagttttgcacattttttaa

SEQ ID NO: 69 - DNA, 58 bp, Artificial Sequence
  Artificially Synthesized
agctttaaaaaatgtgcaaaactgcaaaattcattgtaatagaatgtgtaaggtcaaa

SEQ ID NO: 70 - DNA, 52 bp, Artificial Sequence
  Artificially Synthesized
ttactttctaatgccacagatgcagattacatgtagttattgagaatccttt

SEQ ID NO: 71 - DNA, 58 bp, Artificial Sequence
  Artificially Synthesized
ctagtttactttctaatgccacagatgcagattacatgtagttattgagaatccttta

SEQ ID NO: 72 - DNA, 58 bp, Artificial Sequence
  Artificially Synthesized
agcttaaaggattctcaataactacatgtaatctgcatctgtggcattagaaagtaaa

SEQ ID NO: 73 - DNA, 52 bp, Artificial Sequence
  Artificially Synthesized
agattttatttgtgtggaatgaagtgaggcttgtagtcatggttctagtgtt

SEQ ID NO: 74 - DNA, 58 bp, Artificial Sequence
  Artificially Synthesized
ctagtagattttatttgtgtggaatgaagtgaggcttgtagtcatggttctagtgtta

SEQ ID NO: 75 - DNA, 58 bp, Artificial Sequence
  Artificially Synthesized
agcttaacactagaaccatgactacaagcctcacttcattccacacaaataaaatcta

SEQ ID NO: 76 - DNA, 52 bp, Artificial Sequence
  Artificially Synthesized
tctagtgtttcagtttgccaagtctgtttactgcagtgaaattcatcaaatg

SEQ ID NO: 77 - DNA, 58 bp, Artificial Sequence
  Artificially Synthesized
ctagttctagtgtttcagtttgccaagtctgtttactgcagtgaaattcatcaaatga

SEQ ID NO: 78 - DNA, 58 bp, Artificial Sequence
  Artificially Synthesized
agcttcatttgatgaatttcactgcagtaaacagacttggcaaactgaaacactagaa

SEQ ID NO: 79 - DNA, 51 bp, Artificial Sequence
  Artificially Synthesized
gaaattcatcaaatgtttcagtgtggttttctgtagcctatcatttactgg

SEQ ID NO: 80 - DNA, 57 bp, Artificial Sequence
  Artificially Synthesized
ctagtgaaattcatcaaatgtttcagtgtggttttctgtagcctatcatttactgga

SEQ ID NO: 81 - DNA, 57 bp, Artificial Sequence
  Artificially Synthesized
agcttccagtaaatgataggctacagaaaaccacactgaaacatttgatgaatttca

SEQ ID NO: 82 - DNA, 52 bp, Artificial Sequence
  Artificially Synthesized
ttttccattaaattgccctcatgtcctaatgtgcagtttgtaagtgtgtgtg

SEQ ID NO: 83 - DNA, 58 bp, Artificial Sequence
  Artificially Synthesized
ctagtttttccattaaattgccctcatgtcctaatgtgcagtttgtaagtgtgtgtga

SEQ ID NO: 84 - DNA, 58 bp, Artificial Sequence
  Artificially Synthesized
agcttcacacacacttacaaactgcacattaggacatgagggcaatttaatggaaaaa

SEQ ID NO: 85 - RNA, 22 nt, Homo sapiens
ugaauauaagaacucuugcagu

SEQ ID NO: 86 - RNA, 22 nt, Homo sapiens
caaggacaguguuuuuuguagu

SEQ ID NO: 87 - RNA, 27 nt, Homo sapiens
uuuaaacugccaaauauugacugcagc

SEQ ID NO: 88 - RNA, 22 nt, Homo sapiens
ccugggaggcagagguugcagu

SEQ ID NO: 89 - RNA, 21 nt, Homo sapiens
accugcacuguaagcacuuug

SEQ ID NO: 90 - RNA, 22 nt, Homo sapiens
cuauuacaaugaauuuugcagu

SEQ ID NO: 91 - RNA, 22 nt, Homo sapiens
acagaugcagauuacauguagu

SEQ ID NO: 92 - RNA, 22 nt, Homo sapiens
ggaaugaagugaggcuuguagu

SEQ ID NO: 93 - RNA, 22 nt, Homo sapiens
ugccaagucuguuuacugcagu

SEQ ID NO: 94 - RNA, 22 nt, Homo sapiens
uuucagugugguuuucuguagc

SEQ ID NO: 95 - RNA, 22 nt, Homo sapiens
cccucauguccuaaugugcagu

SEQ ID NO: 96 - RNA, 23 nt, Homo sapiens
augccuguaaucccagcacuuug

SEQ ID NO: 97 - RNA, 23 nt, Homo sapiens
ggauuaauaaagauggcacuuuc

SEQ ID NO: 98 - RNA, 20 nt, Homo sapiens
uacaagugccuucacugcag

SEQ ID NO: 99 - protein, 9 aa, Artificial Sequence
  Artificially Synthesized
AEEADCSKC